SEQUENCE LISTING (51 sequences)

SEQ ID NO: 1 - protein, 560 aa, synthetic construct
MSRKLFASILIGALLGIGAPPSAYAGADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS

SEQ ID NO: 2 - protein, 111 aa, synthetic construct
DIVMTQGTLPNPVPSGESVSITCRSSKSLLYSDGKTYLNWYLQRPGQSPQLLIYWMSTRASGVSDRFSGSGSGTDFTLKISGVEAEDVGIYYCQQGLEFPTFGGGTKLELK

SEQ ID NO: 3 - protein, 120 aa, synthetic construct
EVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSS

SEQ ID NO: 4 - protein, 512 aa, synthetic construct
DIVMTQGTLPNPVPSGESVSITCRSSKSLLYSDGKTYLNWYLQRPGQSPQLLIYWMSTRASGVSDRFSGSGSGTDFTLKISGVEAEDVGIYYCQQGLEFPTFGGGTKLELKGGGGSGGGGSGGGGSEVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSSGGGGSGGGGSGGGGSDIVMTQGTLPNPVPSGESVSITCRSSKSLLYSDGKTYLNWYLQRPGQSPQLLIYWMSTRASGVSDRFSGSGSGTDFTLKISGVEAEDVGIYYCQQGLEFPTFGGGTKLELKGGGGSGGGGSGGGGSEVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSSGGGGS

SEQ ID NO: 5 - protein, 5 aa, synthetic construct
GGGAS

SEQ ID NO: 6 - protein, 13 aa, synthetic construct
GGGASGGGGGGGG

SEQ ID NO: 7 - protein, 17 aa, synthetic construct
GGGASAEAAAKEAAAKA

SEQ ID NO: 8 - protein, 16 aa, synthetic construct
GGGASGTPTPTPTPTG

SEQ ID NO: 9 - protein, 11 aa, synthetic construct
GGGASGAGTGS

SEQ ID NO: 10 - protein, 4 aa, synthetic construct
GTGS

SEQ ID NO: 11 - protein, 11 aa, synthetic construct
GGGGGGGGTGS

SEQ ID NO: 12 - protein, 16 aa, synthetic construct
AEAAAKEAAAKAGTGS

SEQ ID NO: 13 - protein, 14 aa, synthetic construct
GTPTPTPTPTGTGS

SEQ ID NO: 14 - protein, 5 aa, synthetic construct
GGGGS

SEQ ID NO: 15 - protein, 15 aa, synthetic construct
GGGGSGGGGSGGGGS

SEQ ID NO: 16 - protein, 11 aa, synthetic construct
GGGGSHHHHHH

SEQ ID NO: 17 - protein, 25 aa, synthetic construct
GGGGSGGGGSGGGGSHHHHHHHHHH

SEQ ID NO: 18 - protein, 46 aa, synthetic construct
LAEAKVLANRELDKYGVSDFYKRLINKAKTVEGVEALKLHILAALP

SEQ ID NO: 19 - protein, 584 aa, synthetic construct
EAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALA

SEQ ID NO: 20 - protein, 204 aa, synthetic construct
VEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALA

SEQ ID NO: 21 - protein, 1021 aa, synthetic construct
MRFPSIFTAVLFAASSALAAPVNTTTEDETAQIPAEAVIGYSDLEGDFDVAVLPFSNSTNNGLLFINTTIASIAAKEEGVSLEKRAGADDVVDSSKSFVMENFASYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPAKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLPWGGGASGAGTGSDIVMTQGTLPNPVPSGESVSITCRSSKSLLYSDGKTYLNWYLQRPGQSPQLLIYWMSTRASGVSDRFSGSGSGTDFTLKISGVEAEDVGIYYCQQGLEFPTFGGGTKLELKGGGGSGGGGSGGGGSEVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSSGGGGSGGGGSGGGGSDIVMTQGTLPNPVPSGESVSITCRSSKSLLYSDGKTYLNWYLQRPGQSPQLLIYWMSTRASGVSDRFSGSGSGTDFTLKISGVEAEDVGIYYCQQGLEFPTFGGGTKLELKGGGGSGGGGSGGGGSEVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSSGGGGSGGGGSGGGGSHHHHHHHHHH

SEQ ID NO: 22 - protein, 1007 aa, synthetic construct
MRFPSIFTAVLFAASSALAAPVNTTTEDETAQIPAEAVIGYSDLEGDFDVAVLPFSNSTNNGLLFINTTIASIAAKEEGVSLEKRAGADDVVDSSKSFVMENFASYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPAKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLPWGGGASGAGTGSDIVMTQGTLPNPVPSGESVSITCRSSKSLLYSDGKTYLNWYLQRPGQSPQLLIYWMSTRASGVSDRFSGSGSGTDFTLKISGVEAEDVGIYYCQQGLEFPTFGGGTKLELKGGGGSGGGGSGGGGSEVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSSGGGGSGGGGSGGGGSDIVMTQGTLPNPVPSGESVSITCRSSKSLLYSDGKTYLNWYLQRPGQSPQLLIYWMSTRASGVSDRFSGSGSGTDFTLKISGVEAEDVGIYYCQQGLEFPTFGGGTKLELKGGGGSGGGGSGGGGSEVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSSGGGGSHHHHHH

SEQ ID NO: 23 - protein, 1081 aa, synthetic construct
MRFPSIFTAVLFAASSALAAPVNTTTEDETAQIPAEAVIGYSDLEGDFDVAVLPFSNSTNNGLLFINTTIASIAAKEEGVSLEKRAGADDVVDSSKSFVMENFASYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPAKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLPWGGGASGAGTGSLAEAKVLANRELDKYGVSDFYKRLINKAKTVEGVEALKLHILAALPGGGGSGGGGSGTGSDIVMTQGTLPNPVPSGESVSITCRSSKSLLYSDGKTYLNWYLQRPGQSPQLLIYWMSTRASGVSDRFSGSGSGTDFTLKISGVEAEDVGIYYCQQGLEFPTFGGGTKLELKGGGGSGGGGSGGGGSEVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSSGGGGSGGGGSGGGGSDIVMTQGTLPNPVPSGESVSITCRSSKSLLYSDGKTYLNWYLQRPGQSPQLLIYWMSTRASGVSDRFSGSGSGTDFTLKISGVEAEDVGIYYCQQGLEFPTFGGGTKLELKGGGGSGGGGSGGGGSEVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSSGGGGSGGGGSGGGGSHHHHHHHHHH

SEQ ID NO: 24 - protein, 1067 aa, synthetic construct
MRFPSIFTAVLFAASSALAAPVNTTTEDETAQIPAEAVIGYSDLEGDFDVAVLPFSNSTNNGLLFINTTIASIAAKEEGVSLEKRAGADDVVDSSKSFVMENFASYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPAKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLPWGGGASGAGTGSLAEAKVLANRELDKYGVSDFYKRLINKAKTVEGVEALKLHILAALPGGGGSGGGGSGTGSDIVMTQGTLPNPVPSGESVSITCRSSKSLLYSDGKTYLNWYLQRPGQSPQLLIYWMSTRASGVSDRFSGSGSGTDFTLKISGVEAEDVGIYYCQQGLEFPTFGGGTKLELKGGGGSGGGGSGGGGSEVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSSGGGGSGGGGSGGGGSDIVMTQGTLPNPVPSGESVSITCRSSKSLLYSDGKTYLNWYLQRPGQSPQLLIYWMSTRASGVSDRFSGSGSGTDFTLKISGVEAEDVGIYYCQQGLEFPTFGGGTKLELKGGGGSGGGGSGGGGSEVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSSGGGGSHHHHHH

SEQ ID NO: 25 - DNA, 1179 bp, synthetic construct
  source 1..1179: mol_type = other DNA
gctggcgctgacgacgttgttgattcttctaagtccttcgtgatggaaaacttcgcctcctaccacggtactaagccaggttacgttgactccattcagaagggtatccagaagccaaagtccggtactcaaggtaactacgatgatgactggaagggtttctactccaccgacaacaagtacgatgctgctggttactctgtcgacaacgaaaacccattgtctggtaaggctggtggtgttgtcaaggttacttacccaggtttgaccaaggtcttggccttgaaggttgacaacgctgagactatcaagaaagagttgggtttgtccttgaccgagccattgatggaacaagtcggtactgaagagttcatcaagagattcggtgacggtgcctccagagttgttttgtctttgcctttcgctgaaggttcctcatccgtcgagtacattaacaactgggagcaagctaaggccttgtccgttgagttggagatcaacttcgagactagaggtaagaggggtcaagacgctatgtacgagtacatggctcaagcttgtgccggtaacagagttagaagatctgttggttcctccttgtcctgcatcaacttggactgggacgttatcagagacaagactaagaccaagatcgagtccctgaaagagcacggtccaatcaagaacaagatgtctgaatccccagccaagaccgtttctgaagagaaggctaagcagtacctggaagagttccaccaaactgctttggaacacccagaactgtccgagcttaagaccgttactggaactaacccagttttcgctggtgctaactatgctgcttgggctgttaacgttgctcaggttattgactctgagactgccgacaacttggaaaagactactgctgccttgtccatcttgccaggtattggttccgttatgggtattgctgatggtgccgttcaccacaacactgaagaaatcgttgcccagtctatcgccttgtcctccttgatggttgctcaagctattccattggtcggtgagttggttgacattggtttcgctgcctacaacttcgtcgagtccatcattaacctgttccaggttgtccacaactcctacaacagaccagcttactctccaggtcacaagactcaaccatttttgccatgg

SEQ ID NO: 26 - DNA, 333 bp, synthetic construct
  source 1..333: mol_type = other DNA
gacatcgttatgacacaaggtactctgccaaatccagttccatctggtgagtccgtttccattacttgcagatcctccaagtccttgttgtactccgacggtaagacctacttgaactggtacttgcaaagaccaggtcagtccccacagttgttgatctactggatgtctactagagcctccggtgtttctgacagattctctggttctggttccggtactgacttcaccttgaagatttctggtgttgaggctgaggacgtcggtatctactactgtcaacaaggtttggagttcccaaccttcggtggtggtactaagttggagttgaaa

SEQ ID NO: 27 - DNA, 360 bp, synthetic construct
  source 1..360: mol_type = other DNA
gaagttcaattgcaagaatccggtccaggtctggttaagccatctcaatccttgtctttgacctgttccgttaccggttactccatcacctcttcatacaggtggaattggatccgtaagttcccaggtaacagattggagtggatgggttacattaactccgccggtatttccaactacaacccatccttgaagaggcgtatctccatcactagagacacctccaagaaccagttcttcttgcaggttaactccgttactactgaggacgctgccacttactactgtgctagatctgacaacatgggtactaccccattcacttactggggtcagggtactttggttactgtttcctct

SEQ ID NO: 28 - DNA, 1521 bp, synthetic construct
  source 1..1521: mol_type = other DNA
gacatcgttatgacacaaggtactctgccaaatccagttccatctggtgagtccgtttccattacttgcagatcctccaagtccttgttgtactccgacggtaagacctacttgaactggtacttgcaaagaccaggtcagtccccacagttgttgatctactggatgtctactagagcctccggtgtttctgacagattctctggttctggttccggtactgacttcaccttgaagatttctggtgttgaggctgaggacgtcggtatctactactgtcaacaaggtttggagttcccaaccttcggtggtggtactaagttggagttgaaaggtggtggcggatctggtggcggtggaagcggaggtggtggatcagaagttcaattgcaagaatccggtccaggtctggttaagccatctcaatccttgtctttgacctgttccgttaccggttactccatcacctcttcatacaggtggaattggatccgtaagttcccaggtaacagattggagtggatgggttacattaactccgccggtatttccaactacaacccatccttgaagaggcgtatctccatcactagagacacctccaagaaccagttcttcttgcaggttaactccgttactactgaggacgctgccacttactactgtgctagatctgacaacatgggtactaccccattcacttactggggtcagggtactttggttactgtttcctctggcggtggtggctcaggtggcggaggttctggcggaggcggttcagatatagtaatgactcagggaaccctgcctaatcctgtgccttcaggtgaatctgtgtccatcacttgtcgttcctctaagtctctgctgtactctgatggcaagacttaccttaattggtatctgcagaggcctggtcaatctcctcaactgcttatctattggatgagcaccagagcttcaggtgtttccgataggttttctggatccggttctggaaccgactttactttgaaaatctccggtgtcgaagccgaggatgtgggaatctattattgccagcaaggtctggaatttcccactttcggaggtggaacaaagctggaacttaaaggcggcggaggatcaggcggcggtggtagtggtggcggcggtagtgaggttcaacttcaagagtctggtcctggattggtcaagccttcacaatctttgtccctgacttgctccgtcactggctactctattacatcctcttaccgttggaactggataagaaagttccccggaaaccgtcttgaatggatgggctatatcaactctgctggcatctctaattacaaccccagcctgaagcgtaggatctctattaccagagacactagcaagaatcaattcttcctgcaagtcaattccgtcaccactgaagatgctgctacctactattgcgccagatccgataacatgggaaccacaccttttacctactggggacaaggcactctggtgacagtttcatca

SEQ ID NO: 29 - DNA, 15 bp, synthetic construct
  source 1..15: mol_type = other DNA
ggtggtggtgctagc

SEQ ID NO: 30 - DNA, 39 bp, synthetic construct
  source 1..39: mol_type = other DNA
ggtggtggtgctagcggaggtggtggtggtggtggaggt

SEQ ID NO: 31 - DNA, 51 bp, synthetic construct
  source 1..51: mol_type = other DNA
ggtggtggtgctagcgctgaagctgcagctaaggaagctgccgctaaagct

SEQ ID NO: 32 - DNA, 48 bp, synthetic construct
  source 1..48: mol_type = other DNA
ggtggtggtgctagcggtactcctactccaacaccaactcctactggt

SEQ ID NO: 33 - DNA, 33 bp, synthetic construct
  source 1..33: mol_type = other DNA
ggtggtggtgctagcggcgcgggtaccggttct

SEQ ID NO: 34 - protein, 393 aa, synthetic construct
AGADDVVDSSKSFVMENFASYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPAKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLPW

SEQ ID NO: 35 - DNA, 33 bp, synthetic construct
  source 1..33: mol_type = other DNA
ggaggtggtggaggaggtggcggtaccggttct

SEQ ID NO: 36 - DNA, 48 bp, synthetic construct
  source 1..48: mol_type = other DNA
gcagaagctgcagctaaagaagctgctgccaaagccggtaccggttct

SEQ ID NO: 37 - DNA, 42 bp, synthetic construct
  source 1..42: mol_type = other DNA
ggtacacctacaccaactccaacccctacaggtaccggttct

SEQ ID NO: 38 - DNA, 12 bp, synthetic construct
  source 1..12: mol_type = other DNA
ggtaccggttct

SEQ ID NO: 39 - DNA, 48 bp, synthetic construct
  source 1..48: mol_type = other DNA
gctgaagctgccgccaaggaggcagcagcaaaagctggtaccggttct

SEQ ID NO: 40 - DNA, 42 bp, synthetic construct
  source 1..42: mol_type = other DNA
ggtacaccaacacctactccaactcctactggtaccggttct

SEQ ID NO: 41 - DNA, 12 bp, synthetic construct
  source 1..12: mol_type = other DNA
ggtaccggttct

SEQ ID NO: 42 - DNA, 42 bp, synthetic construct
  source 1..42: mol_type = other DNA
ggcggtggcggttctcatcatcatcaccatcactaatctaga

SEQ ID NO: 43 - DNA, 84 bp, synthetic construct
  source 1..84: mol_type = other DNA
ggcggtggcggttctggtggcggtggttctggcggtggcggttcccaccatcaccaccatcatcatcaccatcactaatctaga

SEQ ID NO: 44 - DNA, 138 bp, synthetic construct
  source 1..138: mol_type = other DNA
ttggcggaagctaaagttctggcaaatagagaattggacaagtatggtgttagtgatttctacaaaagattgattaataaagctaaaactgttgaaggtgttgaagctttgaaattgcatattttggctgctttgcct

SEQ ID NO: 45 - DNA, 138 bp, synthetic construct
  source 1..138: mol_type = other DNA
ttagccgaagctaaagttttggctaacagagagcttgacaagtacggtgtttctgatttttataagcgtttaattaacaaggctaagactgttgagggagttgaggctcttaaattgcatatattagctgctcttcct

SEQ ID NO: 46 - DNA, 138 bp, synthetic construct
  source 1..138: mol_type = other DNA
ctagctgaagccaaggtccttgccaatagagaattggataaatacggtgtttctgacttctacaaaagacttataaataaggccaaaactgttgaaggtgttgaggctttgaaactgcatattcttgctgctctacca

SEQ ID NO: 47 - DNA, 138 bp, synthetic construct
  source 1..138: mol_type = other DNA
ttggctgaggctaaggttttggctaacagagaactggacaagtacggtgtctccgacttctacaagagactgatcaacaaggccaagaccgttgaaggtgttgaggctttgaagttgcacatcttggctgctttgcca

SEQ ID NO: 48 - DNA, 1752 bp, synthetic construct
  source 1..1752: mol_type = other DNA
gaggctcataaatctgagattgcccaccgttacaatgacttgggtgaacaacattttaagggtttggtactgattgcctttagtcagtatctgcaaaagtgctcttacgacgaacacgctaaactagtccaagaagtgacagattttgctaaaacttgcgtcgctgatgagagtgctgctaattgcgataaatcccttcatactcttttcggagataaactttgtgctattcctaacctaagagagaattatggtgaactggctgactgttgcacgaagcaagagccagaaagaaacgagtgcttcctgcaacataaggatgacaatccatctttgccaccttttgaaagacctgaagcagaagccatgtgtacgtcatttaaggaaaatccaacaacttttatgggacactaccttcatgaggtagcacgtaggcacccatacttctacgctccagaattgttgtattatgccgaacagtacaatgagattctaacccagtgttgtgctgaggccgataaggaatcctgcttgacccccaagttggatggagttaaagagaaagccttggtttctagtgttagacaaagaatgaaatgcagttctatgcagaaatttggtgaacgagctttcaaggcttgggctgtcgctagattgtctcagacttttcctaacgctgactttgccgagatcactaaactggcaacagatctgacaaaagttaacaaagaatgttgtcatggcgatttgttggaatgtgctgacgatcgtgccgaactggccaaatacatgtgtgaaaatcaagccacgatttcttcaaaattacagacttgttgtgataaacctttattaaagaaagcccactgtttgtccgaggtagaacacgacaccatgccagccgatttacccgcaatcgctgctgattttgtagaggatcaagaggtttgcaaaaattacgctgaggctaaggatgttttccttggcaccttcctgtacgagtactctagaagacatcctgattactccgtctcactgttgctacgattagcaaagaagtacgaagctactcttgaaaaatgttgcgctgaggctaaccccccagcttgttatggtactgtccttgccgaattccagccccttgtggaggagcctaaaaatcttgtaaagacaaattgcgacctatacgagaaactgggagagtatggttttcaaaacgcaattctagttagatacactcaaaaggctcctcaggtttctacgcctaccctggttgaagctgcaagaaaccttggtagagttggcactaaatgctgtaccttgccagaagaccaaagacttccatgcgtcgaggactacttgtccgccatcctgaatagagtgtgcttgttgcatgaaaaaacacctgtttccgaacatgttactaagtgttgttctggtagtcttgtagaaagacgtccctgtttctctgctttgactgtcgatgagacttacgtcccaaaagaattcaaagccgagacctttacttttcattctgatatttgtacccttcctgaaaaggaaaaacaaattaaaaaacaaactgcattagctgaacttgtcaaacataaacctaaagctactgctgagcaattaaaaacagttatggacgacttcgctcaatttctggatacctgttgcaaagctgctgataaagatacttgtttttccactgaaggtcctaatctggtgactagatgtaaagatgctttggct

SEQ ID NO: 49 - DNA, 1752 bp, synthetic construct; identical to SEQ ID NO: 48
  source 1..1752: mol_type = other DNA

SEQ ID NO: 50 - DNA, 1751 bp, synthetic construct
  source 1..1751: mol_type = other DNA
gaggctcacaaatccgagattgcccatagatacaacgacctgggtgaacaacatttcaaaggattagtccttattgctttctctcaatatctgcaaaagtgttcctatgacgaacatgctaaattagttcaagaggttactgatttcgctaagacctgcgttgctgacgaaagtgcagctaattgcgacaaaagtttgcataccttgttcggtgataaactgtgtgctattccaaatttgcgtgagaattacggtgaattagctgattgctgtacgaagcaagagcccgaaaggaacgaatgcttcttgcaacacaaagatgataatccttccctgccaccttttgagagacccgaagccgaagcaatgtgtacttcatttaaagaaaacccaactacatttatgggtcactacttgcacgaggttgctaggagacatccctatttctacgctccagaattattatactacgctgagcaatacaacgaaatcctgactcaatgctgcgccgaagctgataaggaatcttgtctaactcctaagcttgatggagtgaaggagaaggctttggtttcaagtgtgcgtcaaagaatgaaatgttcttccatgcaaaaatttggtgaaagagctttcaaggcatgggcagtagccagattaagtcagacttttcccaacgctgattttgctgaaattaccaaacttgccacggatcttacaaaggttaacaaggagtgttgtcatggtgatttgttagaatgtgcagacgacagggccgagttggcaaagtacatgtgcgagaatcaagctaccattagttccaaacttcagacttgctgtgacaaaccattactgaaaaaggcccactgtttgtcagaggttgagcatgacactatgcctgcagacctaccagctattgctgctgatttcgtggaagaccaagaggtctgtaagaactacgcagaggctaaggatgtcttcttgggtacttttctttacgagtattctaggaggcatccagattattcagtttctctattattgagattagctaaaaagtacgaggccactttggagaaatgctgtgccgaagccaatcctcctgcatgttatggaacagtgttggccgagttccagccattggtggaagaaccaaagaacctggttaaaaccaattgcgatctgtacgaaaagttaggagaatacggcttccagaatgctattttagtgcgttatacacagaaagcccctcaagttagtactcccactctagtcgaagctgctagaaacctgggaagagttggcacaaaatgttgtactttgcccgaggaccaacgtttgccttgcgttgaagattatctatctgccatattgaatcgtgtttgtttgttgcatgagaagactcctgtttctgaacatgtcacaaaatgttgtagtggatccctggttgaaagacgaccttgtttttccgctcttacagttgatgaaacttacgttcctaaggagttcaaggcagaaacctttacttttcacagtgatatttgcacactgcccgaaaaagagaaacagattaaaaagcagaccgctttggctgaattggttaaacataagcctaaggctaccgccgaacaactgaagactgttatggacgacttcgctcaattcctagacacgtgttgtaaagctgctgacaaggacacctgtttttctacagaaggtccaaatctggtcacaagatgtaaggatgctctggc

SEQ ID NO: 51 - DNA, 612 bp, synthetic construct
  source 1..612: mol_type = other DNA
gttgaagagcctaagaatcttgttaagacgaattgcgatttatacgaaaagttaggagagtatggcttccaaaatgcaattcttgtcagatacactcaaaaagctcctcaagtctctactcctactttggtggaagctgctcgtaaccttggaagagtcggtactaaatgctgtactctgcctgaggaccaaagactgccttgcgttgaagactacttgtctgctattttaaacagggtgtgtttgctgcacgaaaaaactccagtgtcagagcatgtgactaaatgctgctccggatctctggtcgaaagaagaccatgtttttcagcccttactgttgacgaaacttatgttcctaaggaatttaaagctgagacctttacctttcactcagatatctgtactttgcctgaaaaggagaagcaaatcaaaaagcaaactgctctggcagaactggttaaacataagccaaaagcaactgcagaacaactgaaaacggtaatggatgatttcgctcagtttttggatacctgctgtaaggctgcagataaggacacttgtttttccacagaaggtccaaatttagttacacgttgtaaagatgccttagca

(19) United States
(12) Patent Application Publication
(10) Pub. No.: US 2024/0285789 A1
(43) Pub. Date: Aug. 29, 2024

(54) Compositions and Methods for Killing PD-1 Positive Cells

(71) Applicant: University of Utah Research Foundation, Salt Lake City, UT (US)

(72) Inventors: Mingnan Chen, Salt Lake City, UT (US); Shuyun Dong, Salt Lake City, UT (US); Tianxiao Zhang, Salt Lake City, UT (US)

(21) Appl. No.: 18/173,680

(22) Filed: Feb. 23, 2023

(51) Int. Cl. A61K 47/68; C07K 14/34; C07K 14/76; C07K 16/28

(52) U.S. Cl. CPC A61K 47/6829; C07K 14/34; C07K 14/76; C07K 16/2818; C07K 2317/622; C07K 2319/01; C07K 2319/30

(57) ABSTRACT

Disclosed are polypeptides comprising a diphtheria toxin and a PD1 targeting moiety. Disclosed are polypeptides comprising, from N- to C-terminus, a truncated diphtheria toxin, and two anti-PD-1 scFvs. Disclosed are nucleic acid constructs comprising a nucleic acid sequence encoding any one of the disclosed polypeptides. Disclosed are nucleic acid constructs comprising a nucleic acid sequence encoding a diphtheria toxin linked to a nucleic acid sequence encoding a PD1 targeting moiety. Disclosed are methods of treating a subject in need thereof comprising administering to the subject a composition comprising one or more of the disclosed polypeptides, vectors, or pharmaceutical compositions.

### STATEMENT REGARDING FEDERALLY SPONSORED RESEARCH

This invention was made with government support under grant AI139535 awarded by the National Institutes of Health. The government has certain rights in this invention.

### REFERENCE TO SEQUENCE LISTING

The Sequence Listing submitted Jun. 23, 2023 as a text file named “21101.0453Ul.xml,” created on Apr. 25, 2023, and having a size of 62,077 bytes is hereby incorporated by reference pursuant to 37 C.F.R. § 1.52(e)(5).

### BACKGROUND

No effective treatments have been found to cure autoimmune diseases (ADs). Severe side effects such as infections can occur after using immunosuppressive drugs for a long time. Programmed Death 1 (PD-1) proteins are inducibly expressed upon activation of lymphocytes (including activated T and B cells) while naïve T and B cells do not express PD-1 protein.

### BRIEF SUMMARY

Disclosed are polypeptides comprising a diphtheria toxin and a PD1 targeting moiety.

Disclosed are polypeptides comprising, from N-to C-terminus, a truncated diphtheria toxin, and two anti-PD-1 scFvs.

Disclosed are nucleic acid constructs comprising a nucleic acid sequence encoding any one of the disclosed polypeptides.

Disclosed are nucleic acid constructs comprising a nucleic acid sequence encoding a diphtheria toxin linked to a nucleic acid sequence encoding a PD1 targeting moiety.

Disclosed are vectors comprising one or more of the disclosed nucleic acid constructions.

Disclosed are recombinant cells comprising one or more of the disclosed nucleic acid constructs, vectors, or polypeptides.

Disclosed are compositions comprising the disclosed polypeptides, nucleic acid constructs and/or vectors.

Disclosed are methods of treating a subject in need thereof comprising administering to the subject a composition comprising one or more of the disclosed polypeptides, vectors, or pharmaceutical compositions.

Disclosed are methods of treating a subject having an autoimmune disease comprising administering to the subject a composition comprising one or more of the disclosed polypeptides, vectors, or pharmaceutical compositions.

Disclosed are methods of treating a subject having cancer comprising administering to the subject a composition comprising one or more of the disclosed polypeptides, vectors, or pharmaceutical compositions.

Disclosed are methods of treating a subject having Type 1 Diabetes comprising administering to the subject a composition comprising one or more of the disclosed polypeptides, vectors, or pharmaceutical compositions.

Disclosed are methods of treating a subject having experimental autoimmune enchephalomyelitis (EAE) comprising administering to the subject a composition comprising one or more of the disclosed polypeptides, vectors, or pharmaceutical compositions.

Disclosed are methods of killing PD-1 positive cells comprising contacting a PD-1 positive cell with a composition comprising a one or more of the disclosed polypeptides, vectors, or pharmaceutical compositions.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows examples of the disclosed polypeptides comprising a His to increase binding ability to the immobilized nickel column. Note: G4S, GGGGS (SEQ ID NO:14) linker V_{H}: Heavy chain variable domain: V_{L}: Light chain variable domain

FIG. 2 shows a schematic illustration of the expression of the immunotoxin in the yeast and purification of the immunotoxin.

FIGS. 3A-C shows the results of transforming the gene of interest into yeast and getting colonies on the plate: selecting single colonies and streaking on the plate to purify yeast: then using SDS-PAGE to monitor the purification step. FIG. 3A shows an expression cassette whereiby the gene of interest was inserted into a Pichia genome. FIG. 3B shows a streak of a single colony on a plate in order to subsequently purify a single colony of the yeast. FIG. 3 shows an SDS-PAGE of the purified yeast. Lane 1 represents the protein ladder: Lane 2 is the concentrated 5 mM imidazole wash flow through of H6 Immunotoxin; lane 3 is the concentrated 5 mM imidazole wash flow through of H10 immunotoxin: after 5 mM imidazole solution wash, beads resuspended by 1 mL of PBS, lane 4 is the PBS-beads mixture of H6 Immunotoxin: lane 5 is the PBS-beads mixture of H10 Immunotoxin; lane 6 is the 20) mM imidazole wash flow through of H6 Immunotoxin; lane 7 is the 20 mM imidazole wash flow through of H10 Immunotoxin: after 20 mM imidazole solution wash, beads resuspended by 1 mL of PBS, lane 8 is the PBS-beads mixture of H6 Immunotoxin: lane 9 is the PBS mixture of H10 Immunotoxin: lane 10 represents the protein ladder.

FIGS. 4A-B shows an example MTS Assay indicating the potency of immunotoxin in specifically eliminating the PD-1 positive cells. FIG. 4A shows a H6 Immunotoxin construct can specifically eliminate PD-1 positive cells with strong potency. FIG. 4B shows H10 Immunotoxin construct shows similar potency in eliminating PD-1 positive tells comparing to H6 Immunotoxin construct.

FIGS. 5A-B show an example Maximum Tolerated Dose (MTD) study indicating the safe doses for mice. FIG. 5A shows data after injection of four doses of 0.125 mg/kg Immunotoxin (total 0.5 mg/kg) into BL6 mice. FIG. 5B shows data after injection of one dose of 0).75 mg/kg immunotoxin (15 ug/mouse) into BL6 mice.

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F. C-D, C-E, and C-F are specifically contemplated and should be considered disclosed from disclosure of A, B, and C: D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C: D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

Headings are provided for convenience only and are not to be construed to limit the invention in any manner. Embodiments illustrated under any heading or in any portion of the disclosure may be combined with embodiments illustrated under the same or any other heading or other portion of the disclosure.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a polypeptide” includes a plurality of such polypeptides, reference to “the linker” is a reference to one or more linkers and equivalents thereof known to those skilled in the art, and so forth.

The expression “operationally linked” or “operably linked” means that the promoter sequence is positioned relative to the coding sequence of the gene of interest such that transcription is able to start. This means that the promoter is positioned upstream of the coding sequence, at a distance enabling the expression of the coding sequence.

The term “percent (%) homology” is used interchangeably herein with the term “percent (%) identity” and refers to the level of nucleic acid or amino acid sequence identity when aligned with a wild type sequence or reference sequence using a sequence alignment program. For example, as used herein, 80% homology means the same thing as 80% sequence identity determined by a defined algorithm, and accordingly a homologue of a given sequence has greater than 80% sequence identity over a length of the given sequence. Exemplary levels of sequence identity include, but are not limited to, 80, 85, 90, 95, 98% or more sequence identity to a given sequence, e.g., the coding sequence for anyone of the inventive polypeptides, as described herein. Exemplary computer programs which can be used to determine identity between two sequences include, but are not limited to, the suite of BLAST programs, e.g., BLASTN, BLASTX, and TBLASTX, BLASTP and TBLASTN, publicly available on the Internet. See also, Altschul, et al., 1990 and Altschul, et al., 1997. Sequence searches are typically carried out using the BLASTN program when evaluating a given nucleic acid sequence relative to nucleic acid sequences in the GenBank DNA Sequences and other public databases. The BLASTX program is preferred for searching nucleic acid sequences that have been translated in all reading frames against amino acid sequences in the GenBank Protein Sequences and other public databases. Both BLASTN and BLASTX are run using default parameters of an open gap penalty of 11.0, and an extended gap penalty of 1.0, and utilize the BLOSUM-62matrix. (See, e.g., Altschul, S. F., et al., Nucleic Acids Res.25:3389-3402, 1997.) A preferred alignment of selected sequences in order to determine“% identity” between two or more sequences, is performed using for example, the CLUSTAL-W program in Mac Vector version 13.0.7, operated with default parameters, including an open gap penalty of 10.0, an extended gap penalty of 0.1, and a BLOSUM 30 similarity matrix.

As used herein, the term “wild-type” refers to a gene or gene product which has the characteristics of that gene or gene product when isolated from a naturally-occurring source.

The terms “variant” and “mutant” are used interchangeably herein. As used herein, the term “mutant” refers to a modified nucleic acid or protein which displays the same characteristics when compared to a reference nucleic acid or protein sequence. A variant can be at least 65, 70, 75, 80, 85, 90, 95, or 99 percent homologues to a reference sequence. In some aspects, a reference sequence can be SEQ ID NO:1. Variants can also include nucleotide sequences that are substantially similar to sequences of miRNA disclosed herein. A “variant” can mean a difference in some way from the reference sequence other than just a simple deletion of an N- and/or C-terminal nucleotide. Variants can also or alternatively include at least one substitution and/or at least one addition, there may also be at least one deletion. Alternatively or in addition, variants can comprise modifications, such as non-natural residues at one or more positions with respect to a reference nucleic acid or protein.

Substitutions, deletions, insertions or any combination thereof may be used to arrive at a final derivative or variant. Generally, these changes are done on a few nucleotides to minimize the alteration of the molecule. However, larger changes may be tolerated in certain circumstances.

Generally, the nucleotide identity between individual variant sequences can be at least 90%, 91%, 92%, 93%, 94%, 95%, 96%, 97%, 98%, 99%, or 100%. Thus, a “variant sequence” can be one with the specified identity to the parent or reference sequence (e.g. wild-type sequence) of the invention, and shares biological function, including, but not limited to, at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent sequence. For example, a “variant sequence” can be a sequence that contains 1, 2, or 3 4 nucleotide base changes as compared to the parent or reference sequence of the invention, and shares or improves biological function, specificity and/or activity of the parent sequence.

Thus, a “variant sequence” can be one with the specified identity to the parent sequence of the invention, and shares biological function, including, but not limited to, at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent sequence. The variant sequence can also share at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of a reference sequence.

The phrase “nucleic acid” as used herein refers to a naturally occurring or synthetic oligonucleotide or polynucleotide, whether DNA or RNA or DNA-RNA hybrid, single-stranded or double-stranded, sense or antisense, which is capable of hybridization to a complementary nucleic acid by Watson-Crick base-pairing. Nucleic acids of the invention can also include nucleotide analogs (e.g., BrdU), and non-phosphodiester internucleoside linkages (e.g., peptide nucleic acid (PNA) or thiodiester linkages). In particular, nucleic acids can include, without limitation, DNA, RNA, cDNA, gDNA, ssDNA, dsDNA or any combination thereof.

By an “effective amount” of a composition as provided herein is meant as a sufficient amount of the composition to provide the desired effect. The exact amount required will vary from subject to subject, depending on the species, age, and general condition of the subject, the severity of disease (or underlying genetic defect) that is being treated, the particular composition used, its mode of administration, and the like. Thus, it is not possible to specify an exact “effective amount.” However, an appropriate “effective amount” may be determined by one of ordinary skill in the art using only routine experimentation.

By “treat” is meant to administer a peptide, nucleic acid, vector, or composition of the invention to a subject, such as a human or other mammal (for example, an animal model), that has an increased susceptibility for developing an autoimmune disease, or that has an autoimmune disease, in order to prevent or delay a worsening of the effects of the disease or condition, or to partially or fully reverse the effects of the disease.

By “prevent” is meant to minimize the chance that a subject who has an increased susceptibility for developing an autoimmune disease will end up with an autoimmune disease.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Polypeptides

Disclosed are polypeptides comprising a diphtheria toxin and a PD1 targeting moiety.

Disclosed are polypeptides comprising, from N-to C-terminus, a truncated diphtheria toxin, and two anti-PD-1 scFvs.

Disclosed are polypeptides comprising, from N-to C-Terminus, a truncated diphtheria toxin, a linker, a variable light chain of an anti-PD1 antibody, a second linker, a variable heavy chain of an anti-PD1 antibody, a third linker, a variable light chain of an anti-PD1 antibody, a forth linker, a variable heavy chain of an anti-PD1 antibody, a fifth linker, and a His tag.

### 1. Diphtheria Toxin

In some aspects, the diphtheria toxin can comprise or consist of the amino acid sequence set forth as

(SEQ ID NO: 1)
MSRKLFASILIGALLGIGAPPSAYAGADDVVDSSKSFVMENFSSYHGTK
PGYVDSIQKGIQKPKSGTQGNYDDDWKGFYSTDNKYDAAGYSVDNENPL
SGKAGGVVKVTYPGLTKVLALKVDNAETIKKELGLSLTEPLMEQVGTEE
FIKRFGDGASRVVLSLPFAEGSSSVEYINNWEQAKALSVELEINFETRG
KRGQDAMYEYMAQACAGNRVRRSVGSSLSCINLDWDVIRDKTKTKIESL
KEHGPIKNKMSESPNKTVSEEKAKQYLEEFHQTALEHPELSELKTVTGT
NPVFAGANYAAWAVNVAQVIDSETADNLEKTTAALSILPGIGSVMGIAD
GAVHHNTEEIVAQSIALSSLMVAQAIPLVGELVDIGFAAYNFVESIINL
FQVVHNSYNRPAYSPGHKTQPFLHDGYAVSWNTVEDSIIRTGFQGESGH
DIKITAENTPLPIAGVLLPTIPGKLDVNKSKTHISVNGRKIRMRCRAID
GDVTFCRPKSPVYVGNGVHANLHVAFHRSSSEKIHSNEISSDSIGVLGY
QKTVDHTKVNSKLSLFFEIKS

(accession number WP_072564851). In some aspects, the diphtheria toxin is a truncated diphtheria toxin. For example, the diphtheria toxin can be a truncated version of SEQ ID NO:1 (Accession Number WP_072564851). In some aspects, the truncated diphtheria toxin comprises or consists of a portion of SEQ ID NO:1. In some aspects, the truncated diphtheria toxin comprises or consists of amino acids from about 1 to about 390 of SEQ ID NO: 1. In some aspects, the truncated diphtheria toxin comprises or consists of amino acids from about 201 to about 384 of SEQ ID NO: 1. In some aspects, the catalytic domain of diphtheria toxin comprises amino acids from about 1 to about 193 of SEQ ID NO: 1. In some aspects, the transolocation domain of diphtheria toxin comprises amino acids from about 1 to about 193 of SEQ ID NO:1. In some aspects, the truncated diphtheria toxin comprises the catalytic domain and translocation domain of diphtheria toxin. For example, the truncated diphtheria toxin can comprise the catalytic domain and translocation domain of SEQ ID NO:1 (Accession Number WP_072564851). In some aspects, the truncated diphtheria toxin comprises at least the catalytic domain. For example, the truncated diphtheria toxin can comprise the catalytic domain of SEQ ID NO: 1 (Accession Number WP_072564851).

In some aspects, the truncated diphtheria toxin comprises, or consists of, the amin acids sequence set forth as

(SEQ ID NO: 34)
AGADDVVDSSKSFVMENFASYHGTKPGYVDSIQKGIQKPKSGTQGNYDD
DWKGFYSTDNKYDAAGYSVDNENPLSGKAGGVVKVTYPGLTKVLALKVD
NAETIKKELGLSLTEPLMEQVGTEEFIKRFGDGASRVVLSLPFAEGSSS
VEYINNWEQAKALSVELEINFETRGKRGQDAMYEYMAQACAGNRVRRSV
GSSLSCINLDWDVIRDKTKTKIESLKEHGPIKNKMSESPAKTVSEEKAK
QYLEEFHQTALEHPELSELKTVTGTNPVFAGANYAAWAVNVAQVIDSET
ADNLEKTTAALSILPGIGSVMGIADGAVHHNTEEIVAQSIALSSLMVAQ
AIPLVGELVDIGFAAYNFVESIINLFQVVHNSYNRPAYSPGHKTQPFLP
W

In some aspects, the diphtheria toxin can be variant of a known diphtheria toxin. In some aspects, the diphtheria toxin can be variant of the diphtheria toxin disclosed herein. In some aspects, a variant can be a sequence that is at least 60, 65, 70, 75, 80, 85, 90, 95, or 99% identical to any of the disclosed sequences of diphtheria toxins.

### 2. PD1 Targeting Moiety

In some aspects, the PD1 targeting moiety is anti-PD1 or a fragment thereof. In some aspects, the anti-PD1 fragment is at least one anti-PD1 scFv. In some aspects, there can be at least one or at least two PD1 targeting moieties. Thus, in some aspects, the PD1 targeting moiety can be at least two anti-PD1 scFvs. In some aspects, an anti-PD1 scFv comprises the variable light chain sequence

DIVMTQGTLPNPVPSGESVSITCRSSKSLLYSDGKTYLNWYLQRPGQSPQLLIYWMST RASGVSDRFSGSGSGTDFTLKISGVEAEDVGIYYCQQGLEFPTFGGGTKLELK (SEQ ID NO:2). In some aspects, an anti-PD1 scFv comprises the sequence
EVQLQESGPGLVKPSQSLSLTCSVTGYSITSSYRWNWIRKFPGNRLEWMGYINSAGIS NYNPSLKRRISITRDTSKNQFFLQVNSVTTEDAATYYCARSDNMGTTPFTYWGQGTL VTVSS (SEQ ID NO:3). In some aspects, when there are at least two PD1 targeting moieties, the at least two PD1 targeting moieties can be identical. In some aspects, the at least two PD1 targeting moieties can be different. In some aspects, when there are at least two PD1 targeting moieties, the at least two PD1 targeting moieties are two anti-PD-1 scFvs. Thus, in some aspects, the two anti-PD-1 scFvs are identical. In some aspects, the two anti-PD-1 scFvs are different. In some aspects, each anti-PD-1 scFv comprises a variable light and a variable heavy chain region.

In some aspects, the two anti-PD1 scFvs comprise, or consist of, the sequence DIVMTQGTLPNPVPSGESVSITCRSSKSLLYSDGKTYLNWYLQRPGQSPQLLIYWMST RASGVSDRFSGSGSGTDFTLKISGVEAEDVGIYYCQQGLEFPTFGGGTKLELKGGGG SGGGGSGGGGSEVQLQESGPGLVKPSQSLSLTCSVTGYSITSSYRWNWIRKFPGNRLEW MGYINSAGISNYNPSLKRRISITRDTSKNQFFLQVNSVTTEDAATYYCARSDNMGTTPFTYW GQGTLVTVSSGGGGSGGGGSGGGGSDIVMTQGTLPNPVPSGESVSITCRSSKSLLYS DGKTYLNWYLQRPGQSPQLLIYWMSTRASGVSDRFSGSGSGTDFTLKISGVEAEDVG IYYCQQGLEFPTFGGGTKLELKGGGGSGGGGSGGGGSEVQLQESGPGLVKPSQSLSL TCSVTGYSITSSYRWNWIRKFPGNRLEWMGYINSAGISNYNPSLKRRISITRDTSKNOFFLOV NSVTTEDAATYYCARSDNMGTTPFTYWGOGTLVTVSSGGGGS (SEQ ID NO:4), wherein the regular sequences are an anti-PD1 variable light chain, the bold sequences are linkers, the italicized sequences are an anti-PD1 variable heavy chain. In some aspects, the linker GGGGS (SEQ ID NO:14) at the C-terminal end can be present or absent.

In some aspects, anti-PD1 antibodies or fragments thereof can be humanized sequences of the sequences described herein.

In some aspects, the PD1 targeting moiety can be variant of a known PD1 targeting moiety. In some aspects, the PD1 targeting moiety can be variant of the PD1 targeting moieties disclosed herein. In some aspects, a variant can be a sequence that is at least 60, 65, 70, 75, 80, 85, 90, 95, or 99% identical to any of the disclosed sequences of PD1 targeting moieties.

### 3. Linkers

In some aspects, the disclosed polypeptides further comprise a linker.

In some aspects, the disclosed polypeptides further comprise a linker between the diphtheria toxin and the PD1 targeting moiety. In some aspects, the disclosed polypeptides further comprise a linker between the truncated diphtheria toxin and the anti-PD-1 scFvs. In some aspects, the linker is GGGAS (SEQ ID NO:5), GGGASGGGGGGGG (SEQ ID NO:6), GGGASAEAAAKEAAAKA (SEQ ID NO:7), GGGASGTPTPTPTPTG (SEQ ID NO:8), GGGASGAGTGS (SEQ ID NO:9), GTGS (SEQ ID NO:10), GGGGGGGGTGS (SEQ ID NO:11), AEAAAKEAAAKAGTGS (SEQ ID NO: 12), or GTPTPTPTPTGTGS (SEQ ID NO:13).

In some aspects, the disclosed polypeptides further comprise one or more linkers within the PD1 targeting moiety. In some aspects, the one or more linkers comprise the sequence GGGGS (SEQ ID NO:14). In some aspects, the one or more linkers comprises GGGGSGGGGSGGGGS (SEQ ID NO:15). In some aspects, the linkers can be between the variable light and variable heavy chain regions of the anti-PD-1 scFvs.

In some aspects, the disclosed polypeptides further comprise a linker between a stabilizing domain and the PD1 targeting moiety. In some aspects, the linker is GTGS (SEQ ID NO: 10), GGGGGGGGTGS (SEQ ID NO:11), AEAAAKEAAAKAGTGS (SEQ ID NO:12), or GTPTPTPTPTGTGS (SEQ ID NO:13).

In some aspects, any of the disclosed linkers can be any of the disclosed linker sequences.

### 4. Detection Moiety

In some aspects, the disclosed polypeptides further comprise a detection moiety. In some aspects, the detection moiety is a histidine (His) tag. In some aspects, the His tag is His₆ or His₁₀. In some aspects, the His tag can be any length. In some aspects, the detection moiety can be any tag that allows for detection and/or isolation of the disclosed polypeptides. For example, the detection tag can not only be a His tag but can also be, but is not limited to, a c-myc tag, a fluorescent tag, a GST tag, a biotin tag, or a FLAG tag.

In some aspects, a 6x-His tag can comprise the sequence GGGGSHHHHHH (SEQ ID NO: 16), wherein the GGGGS (SEQ ID NO:14) is a linker sequence followed by the 6 His residues. In some aspects, a 10x-His tag can comprise the sequence GGGGSGGGGSGGGGSHHHHHHHHHH (SEQ ID NO:17), wherein the GGGGSGGGGSGGGGS (SEQ ID NO:15) is a linker sequence followed by the 10 His residues.

### 5. Stabilizing Protein

In some aspects, the disclosed polypeptides further comprise a stabilizing protein. In some aspects, a stabilizing protein is any protein or fragment thereof that extends the pharmacokinetics of the disclosed polypeptides. For example, in some aspects, a stabilizing protein can be any protein or fragment thereof that extends the half-life of the disclosed polypeptides. For example, the stabilizing protein can by an albumin binding domain, mouse serum albumin, a mouse serum albumin domain III or a portion or fragment thereof.

In some aspects, the stabilizing protein is between the diphtheria toxin and the PD1 targeting moiety. Thus, in some aspects, the polypeptide structure can be diphtheria toxin-stabilizing protein-PD1 targeting moiety. In some aspects, the stabilizing protein is albumin, albumin binding domain, albumin domain III, or a fragment thereof. In some aspects, the albumin binding domain comprises the sequence of LAEAKVLANRELDKYGVSDFYKRLINKAKTVEGVEALKLHILAALP (SEQ ID NO:18). In some aspects, the albumin can be a mouse serum albumin. In some aspects, a mouse serum albumin comprises the sequence of

(SEQ ID NO: 19)
EAHKSEIAHRYNDLGEQHFKGLVLIAFSQYLQKCSYDEHAKLVQEVTDF
AKTCVADESAANCDKSLHTLFGDKLCAIPNLRENYGELADCCTKQEPER
NECFLQHKDDNPSLPPFERPEAEAMCTSFKENPTTFMGHYLHEVARRHP
YFYAPELLYYAEQYNEILTQCCAEADKESCLTPKLDGVKEKALVSSVRQ
RMKCSSMQKFGERAFKAWAVARLSQTFPNADFAEITKLATDLTKVNKEC
CHGDLLECADDRAELAKYMCENQATISSKLQTCCDKPLLKKAHCLSEVE
HDTMPADLPAIAADFVEDQEVCKNYAEAKDVFLGTFLYEYSRRHPDYSV
SLLLRLAKKYEATLEKCCAEANPPACYGTVLAEFQPLVEEPKNLVKTNC
DLYEKLGEYGFQNAILVRYTQKAPQVSTPTLVEAARNLGRVGTKCCTLP
EDQRLPCVEDYLSAILNRVCLLHEKTPVSEHVTKCCSGSLVERRPCFSA
LTVDETYVPKEFKAETFTFHSDICTLPEKEKQIKKQTALAELVKHKPKA
TAEQLKTVMDDFAQFLDTCCKAADKDTCFSTEGPNLVTRCKDALA.

In some aspects, the albumin domain III can be a mouse serum albumin domain III. In some aspects, a mouse serum albumin domain III comprises the sequence of

(SEQ ID NO: 20)
VEEPKNLVKTNCDLYEKLGEYGFQNAILVRYTQKAPQVSTPTLVEAARNL
GRVGTKCCTLPEDQRLPCVEDYLSAILNRVCLLHEKTPVSEHVTKCCSGS
LVERRPCFSALTVDETYVPKEFKAETFTFHSDICTLPEKEKQIKKQTALA
ELVKHKPKATAEQLKTVMDDFAQFLDTCCKAADKDTCFSTEGPNLVTRCK
DALA.

In some aspects, the stabilizing protein can be variant of a known stabilizing protein. In some aspects, the stabilizing protein can be variant of the stabilizing protein disclosed herein. In some aspects, a variant can be a sequence that is at least 60, 65, 70, 75, 80, 85, 90, 95, or 99% identical to any of the disclosed sequences of stabilizing proteins.

### 6. Examples of Specific Polypeptides

Disclosed are polypeptides comprising the sequence of

(SEQ ID NO: 21)
MRFPSIFTAVLFAASSALAAPVNTTTEDETAQIPAEAVIGYSDLEGDFDV
AVLPFSNSTNNGLLFINTTIASIAAKEEGVSLEKRAGADDVVDSSKSFVM
ENFASYHGTKPGYVDSIQKGIQKPKSGTQGNYDDDWKGFYSTDNKYDAAG
YSVDNENPLSGKAGGVVKVTYPGLTKVLALKVDNAETIKKELGLSLTEPL
MEQVGTEEFIKRFGDGASRVVLSLPFAEGSSSVEYINNWEQAKALSVELE
INFETRGKRGQDAMYEYMAQACAGNRVRRSVGSSLSCINLDWDVIRDKTK
TKIESLKEHGPIKNKMSESPAKTVSEEKAKQYLEEFHQTALEHPELSELK
TVTGTNPVFAGANYAAWAVNVAQVIDSETADNLEKTTAALSILPGIGSVM
GIADGAVHHNTEEIVAQSIALSSLMVAQAIPLVGELVDIGFAAYNFVESI
INLFQVVHNSYNRPAYSPGHKTQPFLPWGGGASGAGTGSDIVMTQGT
LPNPVPSGESVSITCRSSKSLLYSDGKTYLNWYLQRPGQSPQLLIYWMS
TRASGVSDRFSGSGSGTDFTLKISGVEAEDVGIYYCQQGLEFPTFGGGT
KLELK [symbol] EVQLQESGPGLVKPSQSLSLTCSVTGYSI
TSSYRWNWIRKFPGNRLEWMGYINSAGISNYNPSLKRRISITRDTSKNQ
FFLQVNSVTTEDAATYYCARSDNMGTTPFTYWGQQGTLVTVSS
GGGGSGGGGSGGGGSDIVMTQGTLPNPVPSGESVSITCRSSKSLLYSDGK
TYLNWYLQRPGQSPQLLIYWMSTRASGVSDRFSGSGSGTDFTLKISGVEA
EDVGIYYCQQGLEFPTFGGGTKLELK [symbol] [symbol] EVQLQESGP
GLVKPSQSLSLTCSVTGYSITSSYRWNWIRKFPGNRLEWMGYINSAGISN
YNPSLKRRISITRDTSKNQFFLQVNSVTTEDAATYYCARSDNMGTTPFTY
WGQGTLVTVSS [symbol] hhhhhhhhhh,

wherein the regular sequence is a truncated diphtheria toxin, the bold sequence is a linker, the underlined sequence is a variable light chain of anti-PD1 scFv, the double underlined sequence is a linker, the italicized and underlined sequence is a variable heavy chain of anti-PD1 scFv, the bold double underlined sequence is a linker, the lowercase letter sequence is a histidine tag.

Disclosed are polypeptides comprising the sequence of

(SEQ ID NO: 22)
MRFPSIFTAVLFAASSALAAPVNTTTEDETAQIPAEAVIGYSDLEGDFDV
AVLPFSNSTNNGLLFINTTIASIAAKEEGVSLEKRAGADDVVDSSKSFVM
ENFASYHGTKPGYVDSIQKGIQKPKSGTQGNYDDDWKGFYSTDNKYDAAG
YSVDNENPLSGKAGGVVKVTYPGLTKVLALKVDNAETIKKELGLSLTEPL
MEQVGTEEFIKRFGDGASRVVLSLPFAEGSSSVEYINNWEQAKALSVELE
INFETRGKRGQDAMYEYMAQACAGNRVRRSVGSSLSCINLDWDVIRDKTK
TKIESLKEHGPIKNKMSESPAKTVSEEKAKQYLEEFHQTALEHPELSELK
TVTGTNPVFAGANYAAWAVNVAQVIDSETADNLEKTTAALSILPGIGSVM
GIADGAVHHNTEEIVAQSIALSSLMVAQAIPLVGELVDIGFAAYNFVESI
INLFQVVHNSYNRPAYSPGHKTQPFLPWGGGASGAGTGSDIVMTQGT
LPNPVPSGESVSITCRSSKSLLYSDGKTYLNWYLQRPGQSPQLLIYWMS
TRASGVSDRFSGSGSGTDFTLKISGVEAEDVGIYYCQQGLEFPTFGGGT
KLELKGGGGSGGGGSGGGGSEVQLQESGPGLVKPSQSLSLTCSVTGYSI
TSSYRWNWIRKFPGNRLEWMGYINSAGISNYNPSLKRRISITRDTSKNQ
FFLQVNSVTTEDAATYYCARSDNMGTTPFTYWGQGTLVTVSS [symbol]
DIVMTQGTLPNPVPSGESVSITCRSSKSLLYSD
GKTYLNWYLQRPGQSPQLLIYWMSTRASGVSDRFSGSGSGTDFTLKISG
VEAEDVGIYYCQQGLEFPTFGGGTKLELKGGGGSGGGGSGGGGSEVQLQ
ESGPGLVKPSQSLSLTCSVTGYSITSSYRWNWIRKFPGNRLEWMGYIN
SAGISNYNPSLKRRISITRDTSKNQFFLQVNSVTTEDAATYYCARSDNM
GTTPFTYWGQGTLVTVSS [symbol] hhhhhh,

wherein the regular sequence is a truncated diphtheria toxin, the bold sequence is a linker, the underlined sequence is a variable light chain of anti-PD1 scFv, the double underlined sequence is a linker, the italicized and underlined sequence is a variable heavy chain of anti-PD1 scFv, the bold double underlined sequence is a linker, the lowercase letter sequence is a histidine tag.

Disclosed are polypeptides comprising the sequence of

(SEQ ID NO: 23)
MRFPSIFTAVLFAASSALAAPVNTTTEDETAQIPAEAVIGYSDLEGDFDV
AVLPFSNSTNNGLLFINTTIASIAAKEEGVSLEKRAGADDVVDSSKSFVM
ENFASYHGTKPGYVDSIQKGIQKPKSGTQGNYDDDWKGFYSTDNKYDAAG
YSVDNENPLSGKAGGVVKVTYPGLTKVLALKVDNAETIKKELGLSLTEPL
MEQVGTEEFIKRFGDGASRVVLSLPFAEGSSSVEYINNWEQAKALSVELE
INFETRGKRGQDAMYEYMAQACAGNRVRRSVGSSLSCINLDWDVIRDKTK
TKIESLKEHGPIKNKMSESPAKTVSEEKAKQYLEEFHQTALEHPELSELK
TVTGTNPVFAGANYAAWAVNVAQVIDSETADNLEKTTAALSILPGIGSVM
GIADGAVHHNTEEIVAQSIALSSLMVAQAIPLVGELVDIGFAAYNFVESI
INLFQVVHNSYNRPAYSPGHKTQPFLPWGGGASGAGTGSLAEAKVLA
NRELDKYGVSDFYKRLINKAKTVEGVEALKLHILAALPGGGGSGGGG
SGTGSDIVMTQGTLPNPVPSGESVSITCRSSKSLLYSDGKTYLNWYL
QRPGQSPQLLIYWMSTRASGVSDRFSGSGSGTDFTLKISGVEAEDVGIYY
CQQGLEFPTFGGGTKLELKGGGGSGGGGSGGGGSEVQLQESGPGLVK
PSQSLSLTCSVTGYSITSSYRWNWIRKFPGNRLEWMGYINSAGISNYNPS
LKRRISITRDTSKNQFFLQVNSVTTEDAATYYCARSDNMGTTPFTYWGQG
TLVTVSSGGGGSGGGGSGGGGSDIVMTQGTLPNPVPSGESVSITCRSS
KSLLYSDGKTYLNWYLQRPGQSPQLLIYWMSTRASGVSDRFSGSGSGTDF
TLKISGVEAEDVGIYYCQQGLEFPTFGGGTKLELKGGGGSGGGGSGGGGS
EVQLQESGPGLVKPSQSLSLTCSVTGYSITSSYRWNWIRKFPGNRLEWMG
YINSAGISNYNPSLKRRISITRDTSKNQFFLQVNSVTTEDAATYYCARSD
NMGTTPFTYWGQGTLVTVSS [symbol] hhhhhhhhhh,

wherein the regular sequence is a truncated diphtheria toxin, the bold sequence is a linker, the italicized sequence is a stabilizing protein, the bold italicized sequence is a linker, the underlined sequence is a variable light chain of anti-PD1 scFv, the double underlined sequence is a linker, the italicized and underlined sequence is a variable heavy chain of anti-PD1 scFv, the lowercase letter sequence is a histidine tag.

(SEQ ID NO: 24)
MRFPSIFTAVLFAASSALAAPVNTTTEDETAQIPAEAVIGYSDLEGDFDV
AVLPFSNSTNNGLLFINTTIASIAAKEEGVSLEKRAGADDVVDSSKSFVM
ENFASYHGTKPGYVDSIQKGIQKPKSGTQGNYDDDWKGFYSTDNKYDAAG
YSVDNENPLSGKAGGVVKVTYPGLTKVLALKVDNAETIKKELGLSLTEPL
MEQVGTEEFIKRFGDGASRVVLSLPFAEGSSSVEYINNWEQAKALSVELE
INFETRGKRGQDAMYEYMAQACAGNRVRRSVGSSLSCINLDWDVIRDKTK
TKIESLKEHGPIKNKMSESPAKTVSEEKAKQYLEEFHQTALEHPELSELK
TVTGTNPVFAGANYAAWAVNVAQVIDSETADNLEKTTAALSILPGIGSVM
GIADGAVHHNTEEIVAQSIALSSLMVAQAIPLVGELVDIGFAAYNFVESI
INLFQVVHNSYNRPAYSPGHKTQPFLPWGGGASGAGTGSLAEAKVLAN
RELDKYGVSDFYKRLINKAKTVEGVEALKLHILAALPGGGGSGGGGSG
TGSDIVMTQGTLPNPVPSGESVSITCRSSKSLLYSDGKTYLNWYLQRPG
QSPQLLIYWMSTRASGVSDRFSGSGSGTDFTLKISGVEAEDVGIYYCQQ
GLEFPTFGGGTKLELK [symbol] EVQLQESGPGLVKPS
QSLSLTCSVTGYSITSSYRWNWIRKFPGNRLEWMGYINSAGISNYNPSLK
RRISITRDTSKNQFFLQVNSVTTEDAATYYCARSDNMGTTPFTYWGQGTL
VTVSS [symbol] DIVMTQGTLPNPVPSGESVSITCRSSK
SLLYSDGKTYLNWYLQRPGQSPQLLIYWMSTRASGVSDRFSGSGSGTDFT
LKISGVEAEDVGIYYCQQGLEFPTFGGGTKLELK [symbol] [symbol]
EVQLQESGPGLVKPSQSLSLTCSVTGYSITSSYRWNW
IRKFPGNRLEWMGYINSAGISNYNPSLKRRISITRDTSKNQFFLQVNSVT
TEDAATYYCARSDNMGTTPFTYWGQGTLVTVSS [symbol] hhhhhh,

wherein the regular sequence is a truncated diphtheria toxin, the bold sequence is a linker, the italicized sequence is a stabilizing protein, the bold italicized sequence is a linker, the underlined sequence is a variable light chain of anti-PD1 scFv, the double underlined sequence is a linker, the italicized and underlined sequence is a variable heavy chain of anti-PD1 scFv, the bold double underlined sequence is a linker, the lowercase letter sequence is a histidine tag.

In some aspects, these same polypeptides are described except for SEQ ID NO:52 is used for the sequence of the truncated diphtheria toxin.

### C. Nucleic Acid Constructs

Disclosed are nucleic acid constructs comprising a nucleic acid sequence capable of encoding any one of the disclosed polypeptides.

Disclosed are nucleic acid constructs comprising a nucleic acid sequence capable of encoding a diphtheria toxin linked to a nucleic acid sequence capable of encoding a PD1 targeting moiety.

Disclosed are nucleic acid constructs comprising, from N-to C-terminus, a nucleic acid sequence capable of encoding a truncated diphtheria toxin and a nucleic acid sequence capable of encoding two anti-PD-1 scFvs.

Disclosed are nucleic acid constructs comprising a nucleic acid sequence capable of encoding a truncated diphtheria toxin, a linker, a variable light chain of an anti-PD1 antibody, a second linker, a variable heavy chain of an anti-PD1 antibody, a third linker, a variable light chain of an anti-PD1 antibody, a forth linker, a variable heavy chain of an anti-PD1 antibody, a fifth linker, and a His tag.

### 1. Diphtheria Toxin

In some aspects, the diphtheria toxin can be a nucleic acid sequence that encodes the protein of SEQ ID NO: 1. In some aspects, the diphtheria toxin is a truncated diphtheria toxin. In some aspects, the truncated diphtheria toxin comprises the catalytic domain and translocation domain of diphtheria toxin. In some aspects, the truncated diphtheria toxin comprises at least the catalytic domain of diphtheria toxin.

In some aspects, the nucleic acid sequence capable of encoding a truncated diphtheria toxin comprises, or consists of, the nucleic acid sequence of

(SEQ ID NO: 25)
GCTGGCGCTGACGACGTTGTTGATTCTTCTAAGTCCTTCGTGATGGAAAA
CTTCGCCTCCTACCACGGTACTAAGCCAGGTTACGTTGACTCCATTCAGA
AGGGTATCCAGAAGCCAAAGTCCGGTACTCAAGGTAACTACGATGATGAC
TGGAAGGGTTTCTACTCCACCGACAACAAGTACGATGCTGCTGGTTACTC
TGTCGACAACGAAAACCCATTGTCTGGTAAGGCTGGTGGTGTTGTCAAGG
TTACTTACCCAGGTTTGACCAAGGTCTTGGCCTTGAAGGTTGACAACGCT
GAGACTATCAAGAAAGAGTTGGGTTTGTCCTTGACCGAGCCATTGATGGA
ACAAGTCGGTACTGAAGAGTTCATCAAGAGATTCGGTGACGGTGCCTCCA
GAGTTGTTTTGTCTTTGCCTTTCGCTGAAGGTTCCTCATCCGTCGAGTAC
ATTAACAACTGGGAGCAAGCTAAGGCCTTGTCCGTTGAGTTGGAGATCAA
CTTCGAGACTAGAGGTAAGAGGGGTCAAGACGCTATGTACGAGTACATGG
CTCAAGCTTGTGCCGGTAACAGAGTTAGAAGATCTGTTGGTTCCTCCTTG
TCCTGCATCAACTTGGACTGGGACGTTATCAGAGACAAGACTAAGACCAA
GATCGAGTCCCTGAAAGAGCACGGTCCAATCAAGAACAAGATGTCTGAAT
CCCCAGCCAAGACCGTTTCTGAAGAGAAGGCTAAGCAGTACCTGGAAGAG
TTCCACCAAACTGCTTTGGAACACCCAGAACTGTCCGAGCTTAAGACCGT
TACTGGAACTAACCCAGTTTTCGCTGGTGCTAACTATGCTGCTTGGGCTG
TTAACGTTGCTCAGGTTATTGACTCTGAGACTGCCGACAACTTGGAAAAG
ACTACTGCTGCCTTGTCCATCTTGCCAGGTATTGGTTCCGTTATGGGTAT
TGCTGATGGTGCCGTTCACCACAACACTGAAGAAATCGTTGCCCAGTCTA
TCGCCTTGTCCTCCTTGATGGTTGCTCAAGCTATTCCATTGGTCGGTGAG
TTGGTTGACATTGGTTTCGCTGCCTACAACTTCGTCGAGTCCATCATTAA
CCTGTTCCAGGTTGTCCACAACTCCTACAACAGACCAGCTTACTCTCCAG
GTCACAAGACTCAACCATTTTTGCCATGG.

In some aspects, the diphtheria toxin can be variant of a known diphtheria toxin. In some aspects, the diphtheria toxin can be variant of the diphtheria toxin disclosed herein. In some aspects, a variant can be a sequence that is at least 60, 65, 70, 75, 80, 85, 90, 95, or 99% identical to any of the disclosed sequences.

In some aspects, the diphtheria toxin encoded by the disclosed nucleic acid constructs can be any of the diphtheria toxins described herein.

### 2. PD1 Targeting Moiety

In some aspects, the PD1 targeting moiety is anti-PD1 or a fragment thereof. In some aspects, the anti-PD1 fragment is at least one anti-PD1 scFv. In some aspects, there can be at least one or at least two PD1 targeting moieties. Thus, in some aspects, the PD1 targeting moiety can be at least two anti-PD1 scFvs. In some aspects, an anti-PD1 scFv is encoded by a nucleic acid sequence having the nucleic acid sequence

GACATCGTTATGACACAAGGTACTCTGCCAAATCCAGTTCCATCTGGTGAGTCCG TTTCCATTACTTGCAGATCCTCCAAGTCCTTGTTGTACTCCGACGGTAAGACCTAC TTGAACTGGTACTTGCAAAGACCAGGTCAGTCCCCACAGTTGTTGATCTACTGGA TGTCTACTAGAGCCTCCGGTGTTTCTGACAGATTCTCTGGTTCTGGTTCCGGTACT GACTTCACCTTGAAGATTTCTGGTGTTGAGGCTGAGGACGTCGGTATCTACTACT GTCAACAAGGTTTGGAGTTCCCAACCTTCGGTGGTGGTACTAAGTTGGAGTTGAA A (SEQ ID NO:26). In some aspects, an anti-PD1 scFv is encoded by a nucleic acid sequence that comprises the nucleic acid sequence of
GAAGTTCAATTGCAAGAATCCGGTCCAGGTCTGGTTAAGCCATCTCAATCCTTGT CTTTGACCTGTTCCGTTACCGGTTACTCCATCACCTCTTCATACAGGTGGAATTGG ATCCGTAAGTTCCCAGGTAACAGATTGGAGTGGATGGGTTACATTAACTCCGCCG GTATTTCCAACTACAACCCATCCTTGAAGAGGCGTATCTCCATCACTAGAGACAC CTCCAAGAACCAGTTCTTCTTGCAGGTTAACTCCGTTACTACTGAGGACGCTGCC ACTTACTACTGTGCTAGATCTGACAACATGGGTACTACCCCATTCACTTACTGGG GTCAGGGTACTTTGGTTACTGTTTCCTCT (SEQ ID NO:27). In some aspects, when there are at least two PD1 targeting moieties, the at least two PD1 targeting moieties can be identical. In some aspects, the at least two PD1 targeting moieties can be different. In some aspects, when there are at least two PD1 targeting moieties, the at least two PD1 targeting moieties are two anti-PD-1 scFvs. Thus, in some aspects, the two anti-PD-1 scFvs are identical. In some aspects, the two anti-PD-1 scFvs are different. In some aspects, each anti-PD-1 scFv comprises a variable light and a variable heavy chain region.

In some aspects, the two anti-PD1 scFvs are encoded by a nucleic acid sequence that comprises, or consists of, the sequence

GACATCGTTATGACACAAGGTACTCTGCCAAATCCAGTTCCATCTGGTGAGTCCG TTTCCATTACTTGCAGATCCTCCAAGTCCTTGTTGTACTCCGACGGTAAGACCTAC TTGAACTGGTACTTGCAAAGACCAGGTCAGTCCCCACAGTTGTTGATCTACTGGA TGTCTACTAGAGCCTCCGGTGTTTCTGACAGATTCTCTGGTTCTGGTTCCGGTACT GACTTCACCTTGAAGATTTCTGGTGTTGAGGCTGAGGACGTCGGTATCTACTACT GTCAACAAGGTTTGGAGTTCCCAACCTTCGGTGGTGGTACTAAGTTGGAGTTGAA AGGTGGTGGCGGATCTGGTGGCGGTGGAAGCGGAGGTGGTGGATCAGAAGT TCAATTGCAAGAATCCGGTCCAGGTCTGGTTAAGCCATCTCAATCCTTGTCTTTGACCT GTTCCGTTACCGGTTACTCCATCACCTCTTCATACAGGTGGAATTGGATCCGTAAGTTC CCAGGTAACAGATTGGAGTGGATGGGTTACATTAACTCCGCCGGTATTTCCAACTACAA CCCATCCTTGAAGAGGOGTATCTCCATCACTAGAGACACCTCCAAGAACCAGTTCTTCT TGCAGGTTAACTCCGTTACTACTGAGGACGCTGCCACTTACTACTGTGCTAGATCTGAC AACATGGGTACTACCCCATTCACTTACTGGGGTCAGGGTACTTTGGTTACTGTTTCCTG TGGCGGTGGTGGCTCAGGTGGCGGAGGTTCTGGCGGAGGCGGTTCAGATAT AGTAATGACTCAGGGAACCCTGCCTAATCCTGTGCCTTCAGGTGAATCTGTGTCC ATCACTTGTCGTTCCTCTAAGTCTCTGCTGTACTCTGATGGCAAGACTTACCTTAA TTGGTATCTGCAGAGGCCTGGTCAATCTCCTCAACTGCTTATCTATTGGATGAGC ACCAGAGCTTCAGGTGTTTCCGATAGGTTTTCTGGATCCGGTTCTGGAACCGACT TTACTTTGAAAATCTCCGGTGTCGAAGCCGAGGATGTGGGAATCTATTATTGCCA GCAAGGTCTGGAATTTCCCACTTTCGGAGGTGGAACAAAGCTGGAACTTAAAGG CGGCGGAGGATCAGGCGGCGGTGGTAGTGGTGGCGGCGGTAGTGAGGTTCA ACTTCAAGAGTCTGGTCCTGGATTGGTCAAGCCTTCACAATCTTTGTCCCTGACTTGCT (CGTCACTGGCTACTCTATTACATCCTCTTACCGTTGGAACTGGATAAGAAAGTTCCCC GGAAACCGTCTTGAATGGATGGGCTATATCAACTCTGCTGGCATCTCTAATTACAACCC CAGCCTGAAGCGTAGGATOTCTATTACCAGAGACACTAGCAAGAATCAATTCTTCCTGC AAGTCAATTCCGTCACCACTGAAGATGCTGCTACCTACTATTGCGCCAGATCCGATAAC ATGGGAACCACACCTTTTACCTACTGGGGACAAGGCACTCTGGTGACAGTTTCATCA (SEQ ID NO:28), wherein the regular sequences are an anti-PD1 variable light chain, the bold sequences are linkers, the italicized sequences are an anti-PD1 variable heavy chain.

In some aspects, the sequence encoding a PD1 targeting moiety can be variant of a known sequence encoding PD1 targeting moiety. In some aspects, the sequence encoding a PD1 targeting moiety can be variant of the sequences encoding PD1 targeting moieties disclosed herein. In some aspects, a variant can be a sequence that is at least 60, 65, 70, 75, 80, 85, 90, 95, or 99% identical to any of the disclosed sequences encoding PD1 targeting moieties.

In some aspects, the PD1 targeting moiety encoded by the disclosed nucleic acid sequences can be any of the PD1 targeting moieties described herein.

### 3. Linkers

In some aspects, the disclosed nucleic acid constructs further comprise a nucleic acid sequence that encodes a linker. The linker can be any of those described herein.

In some aspects, the disclosed nucleic acid constructs further comprise a linker sequence between the sequence that encodes a diphtheria toxin and the sequence that encodes a PD1 targeting moiety. In some aspects, the disclosed nucleic acid constructs further comprise a linker sequence between the sequence that encodes a truncated diphtheria toxin and the sequence that encodes the anti-PD-1 scFvs. In some aspects, the nucleic acid sequence capable of encoding a linker is GGTGGTGGTGCTAGC (SEQ ID NO:29), GGTGGTGGTGCTAGCGGAGGTGGTGGTGGTGGTGGAGGT (SEQ ID NO:30), GGTGGTGGTGCTAGCGCTGAAGCTGCAGCTAAGGAAGCTGCCGCTAAAGCT (SEQ ID NO:31), GGTGGTGGTGCTAGCGGTACTCCTACTCCAACACCAACTCCTACTGGT (SEQ ID NO:32), or GGTGGTGGTGCTAGCGGCGCGGGTACCGGTTCT (SEQ ID NO:33).

In some aspects, the disclosed nucleic acid constructs further comprise a linker sequence between the sequence that encodes a stabilizing domain and the sequence that encodes the PD1 targeting moiety. In some aspects, the nucleic acid sequence capable of encoding a linker is GGTACCGGT,

### GGAGGTGGTGGAGGAGGTGGCGGTACCGGTTCT (SEQ ID NO:35),

### GCAGAAGCTGCAGCTAAAGAAGCTGCTGCCAAAGCCGGTACCGGTTCT (SEQ ID NO:36), GGTACACCTACACCAACTCCAACCCCTACAGGTACCGGTTCT (SEQ ID NO:37), GGTACCGGTTCT (SEQ ID NO:38),

### GCTGAAGCTGCCGCCAAGGAGGCAGCAGCAAAAGCTGGTACCGGTTCT (SEQ ID NO:39), GGTACACCAACACCTACTCCAACTCCTACTGGTACCGGTTCT(SEQ ID NO:40), or GGTACCGGTTCT (SEQ ID NO:41).

In some aspects, the disclosed nucleic acid sequences further comprise one or more linker sequences within the nucleic acid sequences that encode a PD1 targeting moiety. In some aspects, the linker sequences can be between the nucleic acid sequence that encode a variable light and variable heavy chain region of the anti-PD-1 scFvs.

In some aspects, the linkers encoded by the disclosed nucleic acid sequences can be any of the linkers described herein.

### 4. Detection Moiety

In some aspects, the disclosed nucleic acid constructs further comprise a nucleic acid sequence that encodes a detection moiety. In some aspects, the detection moiety is a histidine (His) tag. In some aspects, the His tag is His₆ or His₁₀. In some aspects, the His tag can be any length. In some aspects, the detection moiety can be any tag that allows for detection and/or isolation of the disclosed polypeptides. For example, the detection tag can not only be a His tag but can also be, but is not limited to, a c-myc tag, a fluorescent tag, a GST tag, a biotin tag, or a FLAG tag.

In some aspects, the nucleic acid sequence that encodes a 6x-His tag comprises the sequence GGCGGTGGCGGTTCTCATCATCATCACCATCACTAATCTAGA (SEQ ID NO:42). In some aspects, the nucleic acid sequence that encodes a 10x-His tag comprises the sequence

GGCGGTGGCGGTTCTGGTGGCGGTGGTTCTGGCGGTGGCGGTTCCCACCATCACC ACCATCATCATCACCATCACTAATCTAGA (SEQ ID NO:43). These sequences includes a linker sequence which attaches the His Tag to the rest of the nucleic acid construct.

### 5. Stabilizing Protein

In some aspects, the disclosed nucleic acid constructs further comprise a nucleic acid sequence that encodes a stabilizing protein. In some aspects, a stabilizing protein is any protein or fragment thereof that extends the pharmacokinetics of the disclosed polypeptides. For example, in some aspects, a stabilizing protein is any protein or fragment thereof that extends the half-life of the disclosed polypeptides.

In some aspects, the sequence encoding a stabilizing protein is between the sequence encoding a diphtheria toxin and the sequence encoding a PD1 targeting moiety. Thus, in some aspects, the nucleic acid construct encodes a polypeptide structure that can be diphtheria toxin-stabilizing protein-PD1 targeting moiety. In some aspects, the stabilizing protein is albumin, albumin binding domain, albumin domain III, or a fragment thereof. In some aspects, the nucleic acid sequence capable of encoding an albumin binding domain comprises the sequence of

(SEQ ID NO: 44)
TTGGCGGAAGCTAAAGTTCTGGCAAATAGAGAATTGGACAAGTATGGTGT
TAGTGATTTCTACAAAAGATTGATTAATAAAGCTAAAACTGTTGAAGGTG
TTGAAGCTTTGAAATTGCATATTTTGGCTGCTTTGCCT,
(SEQ ID NO: 45)
TTAGCCGAAGCTAAAGTTTTGGCTAACAGAGAGCTTGACAAGTACGGTGT
TTCTGATTTTTATAAGCGTTTAATTAACAAGGCTAAGACTGTTGAGGGAG
TTGAGGCTCTTAAATTGCATATATTAGCTGCTCTTCCT,
(SEQ ID NO: 46)
CTAGCTGAAGCCAAGGTCCTTGCCAATAGAGAATTGGATAAATACGGTGT
TTCTGACTTCTACAAAAGACTTATAAATAAGGCCAAAACTGTTGAAGGTG
TTGAGGCTTTGAAACTGCATATTCTTGCTGCTCTACCA,
or
(SEQ ID NO: 47)
TTGGCTGAGGCTAAGGTTTTGGCTAACAGAGAACTGGACAAGTACGGTGT
CTCCGACTTCTACAAGAGACTGATCAACAAGGCCAAGACCGTTGAAGGTG
TTGAGGCTTTGAAGTTGCACATCTTGGCTGCTTTGCCA.

In some aspects, the albumin can be a mouse serum albumin. In some aspects, a nucleic acid sequence capable of encoding a mouse serum albumin comprises the sequence of

(SEQ ID NO: 48)
GAGGCTCATAAATCTGAGATTGCCCACCGTTACAATGACTTGGGTGAACAACATT
TTAAGGGTTTGGTACTGATTGCCTTTAGTCAGTATCTGCAAAAGTGCTCTTACGA
CGAACACGCTAAACTAGTCCAAGAAGTGACAGATTTTGCTAAAACTTGCGTCGCT
GATGAGAGTGCTGCTAATTGCGATAAATCCCTTCATACTCTTTTCGGAGATAAAC
TTTGTGCTATTCCTAACCTAAGAGAGAATTATGGTGAACTGGCTGACTGTTGCAC
GAAGCAAGAGCCAGAAAGAAACGAGTGCTTCCTGCAACATAAGGATGACAATCC
ATCTTTGCCACCTTTTGAAAGACCTGAAGCAGAAGCCATGTGTACGTCATTTAAG
GAAAATCCAACAACTTTTATGGGACACTACCTTCATGAGGTAGCACGTAGGCACC
CATACTTCTACGCTCCAGAATTGTTGTATTATGCCGAACAGTACAATGAGATTCT
AACCCAGTGTTGTGCTGAGGCCGATAAGGAATCCTGCTTGACCCCCAAGTTGGAT
GGAGTTAAAGAGAAAGCCTTGGTTTCTAGTGTTAGACAAAGAATGAAATGCAGT
TCTATGCAGAAATTTGGTGAACGAGCTTTCAAGGCTTGGGCTGTCGCTAGATTGT
CTCAGACTTTTCCTAACGCTGACTTTGCCGAGATCACTAAACTGGCAACAGATCT
GACAAAAGTTAACAAAGAATGTTGTCATGGCGATTTGTTGGAATGTGCTGACGAT
CGTGCCGAACTGGCCAAATACATGTGTGAAAATCAAGCCACGATTTCTTCAAAAT
TACAGACTTGTTGTGATAAACCTTTATTAAAGAAAGCCCACTGTTTGTCCGAGGT
AGAACACGACACCATGCCAGCCGATTTACCCGCAATCGCTGCTGATTTTGTAGAG
GATCAAGAGGTTTGCAAAAATTACGCTGAGGCTAAGGATGTTTTCCTTGGCACCT
TCCTGTACGAGTACTCTAGAAGACATCCTGATTACTCCGTCTCACTGTTGCTACG
ATTAGCAAAGAAGTACGAAGCTACTCTTGAAAAATGTTGCGCTGAGGCTAACCC
CCCAGCTTGTTATGGTACTGTCCTTGCCGAATTCCAGCCCCTTGTGGAGGAGCCT
AAAAATCTTGTAAAGACAAATTGCGACCTATACGAGAAACTGGGAGAGTATGGT
TTTCAAAACGCAATTCTAGTTAGATACACTCAAAAGGCTCCTCAGGTTTCTACGC
CTACCCTGGTTGAAGCTGCAAGAAACCTTGGTAGAGTTGGCACTAAATGCTGTAC
CTTGCCAGAAGACCAAAGACTTCCATGCGTCGAGGACTACTTGTCCGCCATCCTG
AATAGAGTGTGCTTGTTGCATGAAAAAACACCTGTTTCCGAACATGTTACTAAGT
GTTGTTCTGGTAGTCTTGTAGAAAGACGTCCCTGTTTCTCTGCTTTGACTGTCGAT
GAGACTTACGTCCCAAAAGAATTCAAAGCCGAGACCTTTACTTTTCATTCTGATA
TTTGTACCCTTCCTGAAAAGGAAAAACAAATTAAAAAACAAACTGCATTAGCTG
AACTTGTCAAACATAAACCTAAAGCTACTGCTGAGCAATTAAAAACAGTTATGG
ACGACTTCGCTCAATTTCTGGATACCTGTTGCAAAGCTGCTGATAAAGATACTTG
TTTTTCCACTGAAGGTCCTAATCTGGTGACTAGATGTAAAGATGCTTTGGCT,
(SEQ ID NO: 49)
GAGGCTCATAAATCTGAGATTGCCCACCGTTACAATGACTTGGGTGAACAACATT
TTAAGGGTTTGGTACTGATTGCCTTTAGTCAGTATCTGCAAAAGTGCTCTTACGA
CGAACACGCTAAACTAGTCCAAGAAGTGACAGATTTTGCTAAAACTTGCGTCGCT
GATGAGAGTGCTGCTAATTGCGATAAATCCCTTCATACTCTTTTCGGAGATAAAC
TTTGTGCTATTCCTAACCTAAGAGAGAATTATGGTGAACTGGCTGACTGTTGCAC
GAAGCAAGAGCCAGAAAGAAACGAGTGCTTCCTGCAACATAAGGATGACAATCC
ATCTTTGCCACCTTTTGAAAGACCTGAAGCAGAAGCCATGTGTACGTCATTTAAG
GAAAATCCAACAACTTTTATGGGACACTACCTTCATGAGGTAGCACGTAGGCACC
CATACTTCTACGCTCCAGAATTGTTGTATTATGCCGAACAGTACAATGAGATTCT
AACCCAGTGTTGTGCTGAGGCCGATAAGGAATCCTGCTTGACCCCCAAGTTGGAT
GGAGTTAAAGAGAAAGCCTTGGTTTCTAGTGTTAGACAAAGAATGAAATGCAGT
TCTATGCAGAAATTTGGTGAACGAGCTTTCAAGGCTTGGGCTGTCGCTAGATTGT
CTCAGACTTTTCCTAACGCTGACTTTGCCGAGATCACTAAACTGGCAACAGATCT
GACAAAAGTTAACAAAGAATGTTGTCATGGCGATTTGTTGGAATGTGCTGACGAT
CGTGCCGAACTGGCCAAATACATGTGTGAAAATCAAGCCACGATTTCTTCAAAAT
TACAGACTTGTTGTGATAAACCTTTATTAAAGAAAGCCCACTGTTTGTCCGAGGT
AGAACACGACACCATGCCAGCCGATTTACCCGCAATCGCTGCTGATTTTGTAGAG
GATCAAGAGGTTTGCAAAAATTACGCTGAGGCTAAGGATGTTTTCCTTGGCACCT
TCCTGTACGAGTACTCTAGAAGACATCCTGATTACTCCGTCTCACTGTTGCTACG
ATTAGCAAAGAAGTACGAAGCTACTCTTGAAAAATGTTGCGCTGAGGCTAACCC
CCCAGCTTGTTATGGTACTGTCCTTGCCGAATTCCAGCCCCTTGTGGAGGAGCCT
AAAAATCTTGTAAAGACAAATTGCGACCTATACGAGAAACTGGGAGAGTATGGT
TTTCAAAACGCAATTCTAGTTAGATACACTCAAAAGGCTCCTCAGGTTTCTACGC
CTACCCTGGTTGAAGCTGCAAGAAACCTTGGTAGAGTTGGCACTAAATGCTGTAC
CTTGCCAGAAGACCAAAGACTTCCATGCGTCGAGGACTACTTGTCCGCCATCCTG
AATAGAGTGTGCTTGTTGCATGAAAAAACACCTGTTTCCGAACATGTTACTAAGT
GTTGTTCTGGTAGTCTTGTAGAAAGACGTCCCTGTTTCTCTGCTTTGACTGTCGAT
GAGACTTACGTCCCAAAAGAATTCAAAGCCGAGACCTTTACTTTTCATTCTGATA
TTTGTACCCTTCCTGAAAAGGAAAAACAAATTAAAAAACAAACTGCATTAGCTG
AACTTGTCAAACATAAACCTAAAGCTACTGCTGAGCAATTAAAAACAGTTATGG
ACGACTTCGCTCAATTTCTGGATACCTGTTGCAAAGCTGCTGATAAAGATACTTG
TTTTTCCACTGAAGGTCCTAATCTGGTGACTAGATGTAAAGATGCTTTGGCT,
or
(SEQ ID NO: 50)
GAGGCTCACAAATCCGAGATTGCCCATAGATACAACGACCTGGGTGAACAACAT
TTCAAAGGATTAGTCCTTATTGCTTTCTCTCAATATCTGCAAAAGTGTTCCTATGA
CGAACATGCTAAATTAGTTCAAGAGGTTACTGATTTCGCTAAGACCTGCGTTGCT
GACGAAAGTGCAGCTAATTGCGACAAAAGTTTGCATACCTTGTTCGGTGATAAAC
TGTGTGCTATTCCAAATTTGCGTGAGAATTACGGTGAATTAGCTGATTGCTGTAC
GAAGCAAGAGCCCGAAAGGAACGAATGCTTCTTGCAACACAAAGATGATAATCC
TTCCCTGCCACCTTTTGAGAGACCCGAAGCCGAAGCAATGTGTACTTCATTTAAA
GAAAACCCAACTACATTTATGGGTCACTACTTGCACGAGGTTGCTAGGAGACATC
CCTATTTCTACGCTCCAGAATTATTATACTACGCTGAGCAATACAACGAAATCCT
GACTCAATGCTGCGCCGAAGCTGATAAGGAATCTTGTCTAACTCCTAAGCTTGAT
GGAGTGAAGGAGAAGGCTTTGGTTTCAAGTGTGCGTCAAAGAATGAAATGTTCT
TCCATGCAAAAATTTGGTGAAAGAGCTTTCAAGGCATGGGCAGTAGCCAGATTA
AGTCAGACTTTTCCCAACGCTGATTTTGCTGAAATTACCAAACTTGCCACGGATC
TTACAAAGGTTAACAAGGAGTGTTGTCATGGTGATTTGTTAGAATGTGCAGACGA
CAGGGCCGAGTTGGCAAAGTACATGTGCGAGAATCAAGCTACCATTAGTTCCAA
ACTTCAGACTTGCTGTGACAAACCATTACTGAAAAAGGCCCACTGTTTGTCAGAG
GTTGAGCATGACACTATGCCTGCAGACCTACCAGCTATTGCTGCTGATTTCGTGG
AAGACCAAGAGGTCTGTAAGAACTACGCAGAGGCTAAGGATGTCTTCTTGGGTA
CTTTTCTTTACGAGTATTCTAGGAGGCATCCAGATTATTCAGTTTCTCTATTATTG
AGATTAGCTAAAAAGTACGAGGCCACTTTGGAGAAATGCTGTGCCGAAGCCAAT
CCTCCTGCATGTTATGGAACAGTGTTGGCCGAGTTCCAGCCATTGGTGGAAGAAC
CAAAGAACCTGGTTAAAACCAATTGCGATCTGTACGAAAAGTTAGGAGAATACG
GCTTCCAGAATGCTATTTTAGTGCGTTATACACAGAAAGCCCCTCAAGTTAGTAC
TCCCACTCTAGTCGAAGCTGCTAGAAACCTGGGAAGAGTTGGCACAAAATGTTGT
ACTTTGCCCGAGGACCAACGTTTGCCTTGCGTTGAAGATTATCTATCTGCCATATT
GAATCGTGTTTGTTTGTTGCATGAGAAGACTCCTGTTTCTGAACATGTCACAAAA
TGTTGTAGTGGATCCCTGGTTGAAAGACGACCTTGTTTTTCCGCTCTTACAGTTGA
TGAAACTTACGTTCCTAAGGAGTTCAAGGCAGAAACCTTTACTTTTCACAGTGAT
ATTTGCACACTGCCCGAAAAAGAGAAACAGATTAAAAAGCAGACCGCTTTGGCT
GAATTGGTTAAACATAAGCCTAAGGCTACCGCCGAACAACTGAAGACTGTTATG
GACGACTTCGCTCAATTCCTAGACACGTGTTGTAAAGCTGCTGACAAGGACACCT
GTTTTTCTACAGAAGGTCCAAATCTGGTCACAAGATGTAAGGATGCTCTGGC.

In some aspects, the nucleic acid sequence capable of encoding an albumin domain III comprises the sequence of

(SEQ ID NO: 51)
GTTGAAGAGCCTAAGAATCTTGTTAAGACGAATTGCGATTTATACGAAAA
GTTAGGAGAGTATGGCTTCCAAAATGCAATTCTTGTCAGATACACTCAAA
AAGCTCCTCAAGTCTCTACTCCTACTTTGGTGGAAGCTGCTCGTAACCTT
GGAAGAGTCGGTACTAAATGCTGTACTCTGCCTGAGGACCAAAGACTGCC
TTGCGTTGAAGACTACTTGTCTGCTATTTTAAACAGGGTGTGTTTGCTGC
ACGAAAAAACTCCAGTGTCAGAGCATGTGACTAAATGCTGCTCCGGATCT
CTGGTCGAAAGAAGACCATGTTTTTCAGCCCTTACTGTTGACGAAACTTA
TGTTCCTAAGGAATTTAAAGCTGAGACCTTTACCTTTCACTCAGATATCT
GTACTTTGCCTGAAAAGGAGAAGCAAATCAAAAAGCAAACTGCTCTGGCA
GAACTGGTTAAACATAAGCCAAAAGCAACTGCAGAACAACTGAAAACGGT
AATGGATGATTTCGCTCAGTTTTTGGATACCTGCTGTAAGGCTGCAGATA
AGGACACTTGTTTTTCCACAGAAGGTCCAAATTTAGTTACACGTTGTAAA
GATGCCTTAGCA.

In some aspects, the nucleic acid sequence encoding a stabilizing protein can be variant of a known stabilizing protein. In some aspects, the stabilizing protein can be variant of the stabilizing protein disclosed herein. In some aspects, a variant can be a sequence that is at least 60, 65, 70, 75, 80, 85, 90, 95, or 99% identical to any of the disclosed nucleic acid sequences that encode stabilizing proteins.

### D. Vectors

Disclosed are vectors comprising one or more of the disclosed nucleic acid constructions.

Disclosed are vectors comprising a nucleic acid construct comprising a nucleic acid sequence encoding a diphtheria toxin linked to a nucleic acid sequence encoding a PD1 targeting moiety.

Disclosed are vectors comprising a nucleic acid construct comprising, from N-to C-terminus, a nucleic acid sequence encoding a truncated diphtheria toxin and a nucleic acid sequence encoding two anti-PD-1 scFvs.

In some aspects, the vector is an expression vector. The term “expression vector” includes any vector, (e.g., a plasmid, cosmid or phage chromosome) containing a gene construct in a form suitable for expression by a cell (e.g., linked to a transcriptional control element). “Plasmid” and “vector” are used interchangeably, as a plasmid is a commonly used form of vector. Moreover, the invention is intended to include other vectors which serve equivalent functions.

In some aspects, a vector can further comprise a promoter operably linked to the nucleic acid sequence encoding a diphtheria toxin In some aspects, any of the disclosed vectors can comprise any of the following vector features.

There are a number of compositions and methods which can be used to deliver the disclosed nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

Expression vectors can be any nucleotide construction used to deliver genes or gene fragments into cells (e.g., a plasmid), or as part of a general strategy to deliver genes or gene fragments, e.g., as part of recombinant retrovirus or adenovirus (Ram et al. Cancer Res. 53:83-88, (1993)). For example, disclosed herein are expression vectors comprising a nucleic acid construct comprising a nucleic acid sequence capable of encoding a diphtheria toxin linked to a nucleic acid sequence capable of encoding a PD1 targeting moiety.

The “control elements” present in an expression vector are those non-translated regions of the vector—enhancers, promoters, 5′ and 3′ untranslated regions—which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the pBLUESCRIPT phagemid (Stratagene, La Jolla, Calif.) or pSPORTI plasmid (Gibco BRL, Gaithersburg, Md.) and the like may be used. If it is necessary to generate a cell line that contains multiple copies of the sequence encoding a polypeptide, vectors based on SV40 or EBV may be advantageously used with an appropriate selectable marker.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell Bio. 4: 1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, a-fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

In some aspects, a promoter can be regulatable. The promoter or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

Optionally, the promoter or enhancer region can act as a constitutive promoter or enhancer to maximize expression of the polynucleotides of the invention. In certain constructs the promoter or enhancer region can be active in all eukaryotic cell types, even if it is only expressed in a particular type of cell at a particular time.

In some aspects, a vector comprises one or more pol promoters, one or more pol promoters II, one or more pol III promoters, or combinations thereof. Examples of pol II promoters include, but are not limited to the retroviral Rous sarcoma virus (RSV) LTR promoter (optionally with the RSV enhancer), the cytomegalovirus (CMV) promoter (optionally with the CMV enhancer), the SV40 promoter, the dihydrofolate reductase promoter, the B-actin promoter, the phospho glycerol kinase (PGK) promoter, and the EFla promoter. In some aspects, pol II promoters can be engineered to confer tissue specific and inducible regulation of gRNAs. Examples of pol III promoters include, but are not limited to, U6 and Hl promoters. In an aspect, the promoter is U6. In an aspect, the promoter operably linked to the gRNA is a Pol III promoter, human u6, mouse U6, H1, or 7SK. Examples of promoters can be those derived from polyoma, adenovirus 2, cytomegalovirus, simian virus 40), and others disclosed herein and known in the art.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contains a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases.

The expression vectors can include a nucleic acid sequence encoding a marker product. This marker product can be used to determine if the gene has been delivered to the cell and once delivered is being expressed. Marker genes can include, but are not limited to the E. coli lacZ gene, which encodes ß-galactosidase, and the gene encoding the green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

Another type of selection that can be used with the composition and methods disclosed herein is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin. (Southern P. and Berg. P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

As used herein, plasmid or viral vectors are agents that transport the disclosed nucleic acids, such as a nucleic acid sequence capable of encoding one or more of the disclosed peptides into the cell without degradation and include a promoter yielding expression of the gene in the cells into which it is delivered. In some embodiments the nucleic acid sequences disclosed herein are derived from either a virus or a retrovirus. Viral vectors are, for example, Adenovirus, Adeno-associated virus, Herpes virus, Vaccinia virus, Polio virus, lentivirus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors can have higher transaction abilities (i.e., ability to introduce genes) than chemical or physical methods of introducing genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

Retroviral vectors, in general, are described by Verma, I. M., Retroviral vectors for gene transfer. In Microbiology, Amer. Soc, for Microbiology, pp. 229-232. Washington, (1985), which is hereby incorporated by reference in its entirety. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)): the teachings of which are incorporated herein by reference in their entirety for their teaching of methods for using retroviral vectors for gene therapy.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serves as the priming site for the synthesis of the second strand of DNA synthesis, and specific sequences near the ends of the LTRs that enable the insertion of the DNA state of the retrovirus to insert into the host genome. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

The construction of replication-defective adenoviruses has been described (Berkner et al., J. Virology 61:1213-1220 (1987); Massie et al., Mol. Cell. Biol. 6:2872-2883 407641069 30) (1986); Haj-Ahmad et al., J. Virology 57:267-274 (1986): Davidson et al., J. Virology 61:1226-1239 (1987): Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993): Roessler, J. Clin. Invest. 92:1085-1092 (1993): Moullier, Nature Genetics 4:154-159 (1993): La Salle, Science 259:988-990 (1993); Gomez-Foix, J. Biol. Chem. 267:25129-25134 (1992): Rich, Human Gene Therapy 4:461-476 (1993): Zabner, Nature Genetics 6:75-83 (1994): Guzman, Circulation Research 73:1201-1207 (1993): Bout, Human Gene Therapy 5:3-10 (1994): Zabner, Cell 75:207-216 (1993); Caillaud, Eur. J. Neuroscience 5:1287-1291 (1993); and Ragot, J. Gen. Virology 74:501-507 (1993)) the teachings of which are incorporated herein by reference in their entirety for their teaching of methods for using retroviral vectors for gene therapy. Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973): Svensson and Persson, J. Virology 55:442-449 (1985): Seth, et al., J. Virol. 51:650-655 (1984); Seth, et al., Mol. Cell. Biol., 4:1528-1533 (1984): Varga et al., J. Virology 65:6061-6070 (1991): Wickham et al., Cell 73:309-319 (1993)).

A viral vector can be one based on an adenovirus which has had the E1 gene removed and these virons are generated in a cell line such as the human 293 cell line. Optionally, both the E1 and E3 genes are removed from the adenovirus genome.

Another type of viral vector that can be used to introduce the polynucleotides of the invention into a cell is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, CA, which can contain the herpes simplex virus thymidine kinase gene, HSV-tk, or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus. Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference in its entirety for material related to the AAV vector.

The inserted genes in viral and retroviral vectors usually contain promoters, or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

Other useful systems include, for example, replicating and host-restricted non-replicating vaccinia virus vectors. In addition, the disclosed nucleic acid sequences can be delivered to a target cell in a non-nucleic acid based system. For example, the disclosed polynucleotides can be delivered through electroporation, or through lipofection, or through calcium phosphate precipitation. The delivery mechanism chosen will depend in part on the type of cell targeted and whether the delivery is occurring for example in vivo or in vitro.

### E. Recombinant Cells

Disclosed are recombinant cells comprising one or more of the disclosed nucleic acid constructs, vectors, or polypeptides. For example, disclosed are recombinant cells comprising a nucleic acid construct, wherein the nucleic acid construct comprises a nucleic acid sequence encoding a diphtheria toxin linked to a nucleic acid sequence encoding a PD1 targeting moiety. Also disclosed are recombinant cells comprising a polypeptide comprising a diphtheria toxin and a PD1 targeting moiety.

In some aspects, the cell is a mammalian cell.

### F. Compositions

Disclosed are compositions comprising the disclosed polypeptides, nucleic acid constructs and/or vectors. Disclosed are compositions comprising a nucleic acid construct, wherein the nucleic acid construct comprises a nucleic acid sequence encoding any one of the disclosed polypeptides. Also disclosed are compositions comprising a polypeptide comprising a diphtheria toxin and a PD1 targeting moiety.

The disclosed compositions can further comprise a pharmaceutically acceptable carrier. Thus, disclosed are pharmaceutical compositions.

### 1. Delivery of Compositions

In the methods described herein, delivery (or administration) of the compositions to cells can be via a variety of mechanisms. As defined above, disclosed herein are compositions comprising any one or more of the polypeptides, nucleic acids, and/or vectors described herein can be used to produce a composition which can also include a carrier such as a pharmaceutically acceptable carrier. For example, disclosed are pharmaceutical compositions, comprising the peptides disclosed herein, and a pharmaceutically acceptable carrier.

For example, the compositions described herein can comprise a pharmaceutically acceptable carrier. By “pharmaceutically acceptable” is meant a material or carrier that would be selected to minimize any degradation of the active ingredient and to minimize any adverse side effects in the subject, as would be well known to one of skill in the art. Examples of carriers include dimyristoylphosphatidyl (DMPC), phosphate buffered saline or a multivesicular liposome. For example, PG:PC:Cholesterol: peptide or PC:peptide can be used as carriers in this invention. Other suitable pharmaceutically acceptable carriers and their formulations are described in Remington: The Science and Practice of Pharmacy (19th ed.) ed. A. R. Gennaro, Mack Publishing Company, Easton, P A 1995. Typically, an appropriate amount of pharmaceutically-acceptable salt is used in the formulation to render the formulation isotonic. Other examples of the pharmaceutically-acceptable carrier include, but are not limited to, saline, Ringer's solution and dextrose solution. The pH of the solution can be from about 5 to about 8, or from about 7 to about 7.5. Further carriers include sustained release preparations such as semi-permeable matrices of solid hydrophobic polymers containing the composition, which matrices are in the form of shaped articles, e.g., films, stents (which are implanted in vessels during an angioplasty procedure), liposomes or microparticles. It will be apparent to those persons skilled in the art that certain carriers may be more preferable depending upon, for instance, the route of administration and concentration of composition being administered. These most typically would be standard carriers for administration of drugs to humans, including solutions such as sterile water, saline, and buffered solutions at physiological pH.

Pharmaceutical compositions can also include carriers, thickeners, diluents, buffers, preservatives and the like, as long as the intended activity of the polypeptide, peptide, nucleic acid, vector of the invention is not compromised. Pharmaceutical compositions may also include one or more active ingredients (in addition to the composition of the invention) such as antimicrobial agents, anti-inflammatory agents, anesthetics, and the like. The pharmaceutical composition may be administered in a number of ways depending on whether local or systemic treatment is desired, and on the area to be treated.

Preparations of parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for optical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids, or binders may be desirable. Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mon-, di-, trialkyl and aryl amines and substituted ethanolamines.

The disclosed delivery techniques can be used not only for the disclosed compositions but also the disclosed polypeptides, nucleic acid constructs and vectors.

### G. Dosages

Disclosed are dosing regimens comprising administering a single dose of one or more of the disclosed compositions or polypeptides to a subject in need thereof, wherein the single dose comprises an amount effective to kill PD1+ cells.

Disclosed are dosing regimens comprising administering at least two doses of one or more of the disclosed compositions or polypeptides to a subject in need thereof, wherein each dose is the same concentration. In some aspects, each dose after a first dose can be decreased. In some aspects, each dose after a first dose can be increased.

In some aspects, a single dose can be a continuous administration. In some aspects, a continuous administration can be hours, days, weeks, or months. In some aspects, there can be two or more doses. In some aspects, the two or more doses can be administered days, weeks, or months apart.

In some aspects, a dose for mice can be 0.15 mg/kg. Thus, an equivalent dose for humans can be determined.

### H. Methods of Treating

Disclosed are methods of treating a subject in need thereof comprising administering to the subject a composition comprising one or more of the disclosed polypeptides, vectors, or pharmaceutical compositions. In some aspects, a subject in need thereof can be a subject having a disease or disorder with increased PD1+ cells. In some aspects, the disclosed methods can be protein therapies or gene therapies.

Disclosed are methods of treating a subject having an autoimmune disease comprising administering to the subject a composition comprising one or more of the disclosed polypeptides, vectors, or pharmaceutical compositions.

Disclosed are methods of treating a subject having cancer comprising administering to the subject a composition comprising one or more of the disclosed polypeptides, vectors, or pharmaceutical compositions.

Disclosed are methods of treating a subject having Type 1 Diabetes comprising administering to the subject a composition comprising one or more of the disclosed polypeptides, vectors, or pharmaceutical compositions.

Disclosed are methods of treating a subject having experimental autoimmune enchephalomyelitis (EAE) comprising administering to the subject a composition comprising one or more of the disclosed polypeptides, vectors, or pharmaceutical compositions.

In some aspects, administering comprises a dosing regimen of at least one, two, three, four, or five doses of the composition. In some aspects, each dose is 0.15 mg/kg.

In some aspects, the doses are given at least 24 hours apart from each other. In some aspects, the doses are given at least 48 hours apart from each other.

In some aspects, administering occurs intraperitoneally and/or intravenously.

In some aspects, the disclosed methods result in PD-1 positive cells being killed.

### I. Methods of Killing PD1+ Cells

Disclosed are methods of killing PD-1 positive cells comprising contacting a PD-1 positive cell with a composition comprising a one or more of the disclosed polypeptides, vectors, or pharmaceutical compositions.

In some aspects, contacting a PD-1 positive cell with a composition or pharmaceutical composition occurs in vivo or in vitro.

In some aspects, any of the doses described herein can be used to kill PD-1 positive cells.

### J. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits comprising one or more of the disclosed polypeptides, nucleic acid sequences, vectors, or recombinant cells. The kits also can contain instructions for making or using the disclosed polypeptides, nucleic acid sequences, vectors, or recombinant cells.

### EXAMPLES

### A. Recombinant Anti-PD-1-Immunotoxin for Autoimmune Disease Treatment

No effective treatments have been found to cure autoimmune diseases (ADs): new therapies are in urgent need to investigate. Severe side effects such as infections can occur after using immunosuppressive drugs for a long time. Programmed Death 1 (PD-1) proteins are inducibly expressed upon activation of lymphocytes (including activated T and B cells) while naïve T and B cells do not express PD-1 protein.

The study described herein shows that an anti-PD-1 immunotoxin possesses selective toxicity to PD-1 positive cells, which can be used as an approach to ameliorate autoimmune diseases.

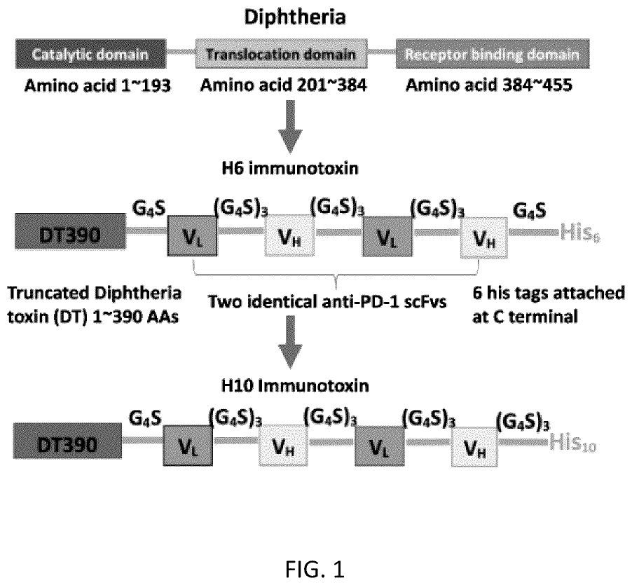

FIG. 1 shows an example of an immunotoxin design to specifically eliminate PD-1 positive immune cells in the ADs mice model: modified constructs can be used to get a better yield. The modified construct with 10 his tags attached aims to increase the binding ability to the immobilized nickel column thus reducing the immunotoxin loss during the purification process.

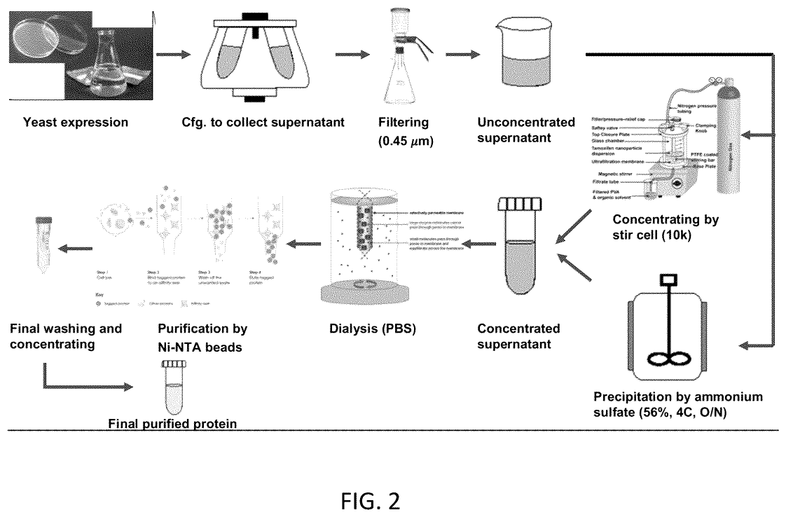

FIG. 2 shows the yeast expression and purification process of one of the disclosed polypeptides.

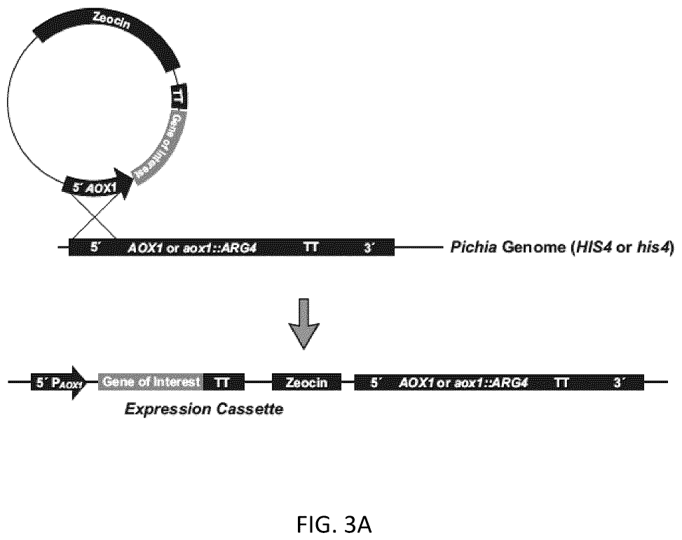

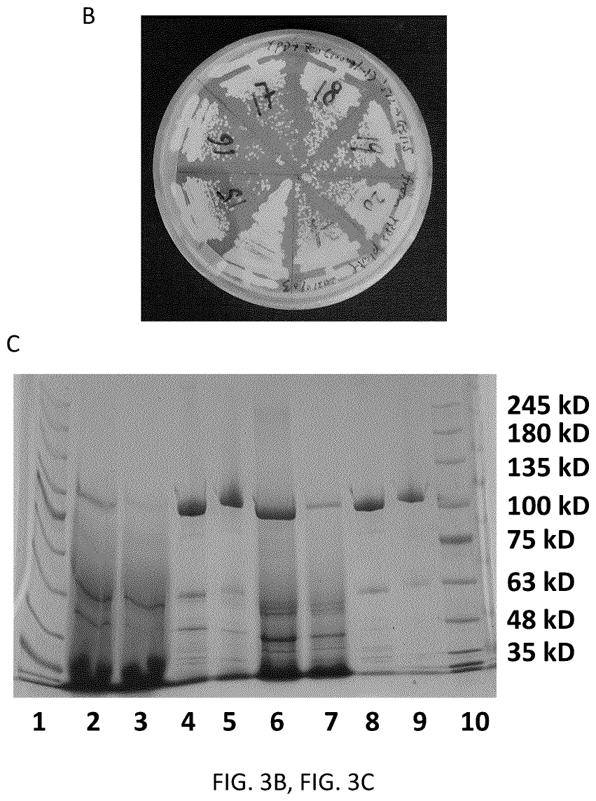

FIGS. 3A-3C show an example yeast construct, plating colonies and then examining the purification process on an SDS-PAGE. FIG. 3C shows the results of an SDS-PAGE; Lane 1 represents the protein ladder: Lane 2 is the concentrated 5 mM imidazole wash flow through of H6 Immunotoxin: lane 3 is the concentrated 5 mM imidazole wash flow through of H10 immunotoxin: after 5 mM imidazole solution wash, beads resuspended by 1 mL of PBS, lane 4 Is the PBS-beads mixture of H6 Immunotoxin; lane 5 is the PBS-beads mixture of H10 Immunotoxin: lane 6 is the 20 mM imidazole wash flow through of H6 Immunotoxin: lane 7 is the 20 mM imidazole wash flow through of H10 Immunotoxin: after 20 mM imidazole solution wash, beads resuspended by 1 mL of PBS, lane 8 is the PBS-beads mixture of H6 Immunotoxin: lane 9 is the PBS mixture of H10 Immunotoxin: lane 10 represents the protein ladder.

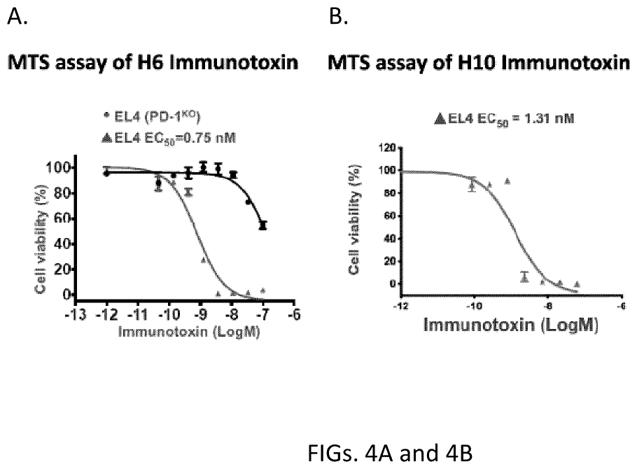

FIGS. 4A-4B show that an H6 immunotoxin construct can specifically eliminate PD-1 positive cells with strong potency. An H10 immunotoxin construct shows similar potency in eliminating PD-1 positive cells comparing to H6 Immunotoxin construct.

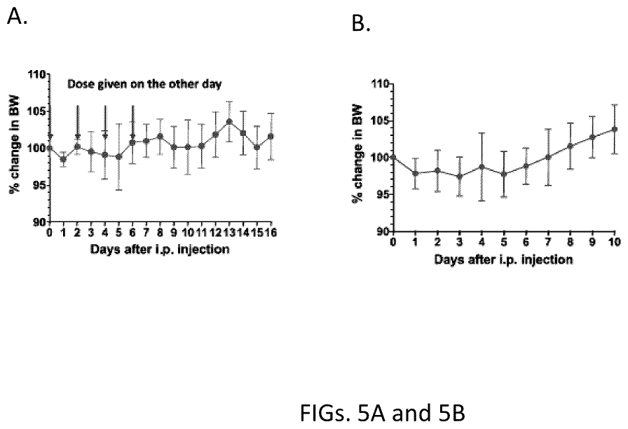

After injection of four doses of 0.125 mg/kg Immunotoxin (total 0.5 mg/g), the steady body weight of BL6 mice indicates that they are safe serial doses (FIG. 5A). After injection of one dose of 0.75 mg/kg Immunotoxin (15 ug/mouse), the steady body weight of BL6 mice indicates that it is a safe dose (FIG. 5B).

No dehydration, loss of muscularity, signs of unhealthy like less movement and no response, lethargy, inappetence, loss of body weight, and mortality was observed during the process.

This study shows that an H10 immunotoxin shows significantly stronger binding to a nickel column than H6 Immunotoxin, both H6 immunotoxin and H10 immunotoxin show potent cytotoxicity in killing the PD-1 positive cells, and an MTD study indicates that 0.75 mg/kg is a safe dose for BL6 mice.

### B. Animal Studies

Several animal models can be used to study the effects of the disclosed compositions.

For cancer treatment, 2×10⁴ EL4 (PD-1⁺ tumor lymphoma cells) can be transferred to the mice through intravenous (i.v.) injection on Day 0. Then starting on day 1, 5 doses of immunotoxin i.v. can be given every other day for treatment, and each dose is 0.15 mg/kg.

For immune checkpoint accelerated type 1 diabetes (TID) model, starting on day 0, 5 doses of immunotoxin treatment can be given to NOD mice through intraperitoneal (i.p.) injection every other day, and each dose is 0.15 mg/kg. Then on day 10, the mice can be treated with 5 doses of anti-PD-1 blocking antibody every other day to accelerate the hyperglycemia, and each dose is 10 mg/kg.

For a spontaneous TID model, the NOD mice can be treated with 0.15 mg/kg immunotoxin weekly and blood glucose levels are monitored.

For EAE treatment, on day 0, mice can be treated with 4 subcutaneous injections with a total of 50 ug proteolipid protein (PLP) on the back of SJL/J mice. On day 3, 5 doses of immunotoxin are given through i.p. every other day, and each dose is 0.15 mg/kg.

Those skilled in the art will recognize, or be able to ascertain using no more than routine experimentation, many equivalents to the specific embodiments of the method and compositions described herein. Such equivalents are intended to be encompassed by the following claims.

1.-8. (canceled)

9. A polypeptide comprising a diphtheria toxin and a PD1 targeting moiety.

10. The polypeptide of claim 9, wherein the PD1 targeting moiety is anti-PD1 or a fragment thereof.

11. The polypeptide of claim 9, wherein the diphtheria toxin is a truncated diphtheria toxin.

12. The polypeptide of claim 9, wherein the truncated diphtheria toxin comprises the catalytic domain and translocation domain of diphtheria toxin.

13. The polypeptide of claim 9, wherein the anti-PD1 fragment is at least one anti-PD1 scFv.

14. The polypeptide of claim 13, wherein the anti-PD1 scFv comprises the sequence
(SEQ ID NO: 2)
DIVMTQGTLPNPVPSGESVSITCRSSKSLLYSDGKTYLNWYLQRPGQSPQ
LLIYWMSTRASGVSDRFSGSGSGTDFTLKISGVEAEDVGIYYCQQGLEFP
TFGGGTKLELK,
or
(SEQ ID NO: 3)
EVQLQESGPGLVKPSQSLSLTCSVTGYSITSSYRWNWIRKFPGNRLEWMG
YINSAGISNYNPSLKRRISITRDTSKNQFFLQVNSVTTEDAATYYCARSD
NMGTTPFTYWGQGTLVTVSS.

15. (canceled)

16. The polypeptide of claim 9, further comprising a linker between the diphtheria toxin and the PD1 targeting moiety.

17. (canceled)

18. The polypeptide of claim 9, further comprising one or more linkers in the PD1 targeting moiety.

19. (canceled)

20. (canceled)

21. (canceled)

22. (canceled)

23. The polypeptide of claim 9, further comprises a stabilizing protein.

24. (canceled)

25. The polypeptide of claim 23, wherein the stabilizing protein is albumin, albumin binding domain, albumin domain III, or a fragment thereof.

26. The polypeptide of claim 25, wherein the stabilizing protein comprises the sequence of
(SEQ ID NO: 18)
LAEAKVLANRELDKYGVSDFYKRLINKAKTVEGVEALKLHILAALP,
(SEQ ID NO: 19)
EAHKSEIAHRYNDLGEQHFKGLVLIAFSQYLQKCSYDEHAKLVQEVTDFA
KTCVADESAANCDKSLHTLFGDKLCAIPNLRENYGELADCCTKQEPERNE
CFLQHKDDNPSLPPFERPEAEAMCTSFKENPTTFMGHYLHEVARRHPYFY
APELLYYAEQYNEILTQCCAEADKESCLTPKLDGVKEKALVSSVRQRMKC
SSMQKFGERAFKAWAVARLSQTFPNADFAEITKLATDLTKVNKECCHGDL
LECADDRAELAKYMCENQATISSKLQTCCDKPLLKKAHCLSEVEHDTMPA
DLPAIAADFVEDQEVCKNYAEAKDVFLGTFLYEYSRRHPDYSVSLLLRLA
KKYEATLEKCCAEANPPACYGTVLAEFQPLVEEPKNLVKTNCDLYEKLGE
YGFQNAILVRYTQKAPQVSTPTLVEAARNLGRVGTKCCTLPEDQRLPCVE
DYLSAILNRVCLLHEKTPVSEHVTKCCSGSLVERRPCFSALTVDETYVPK
EFKAETFTFHSDICTLPEKEKQIKKQTALAELVKHKPKATAEQLKTVMDD
FAQFLDTCCKAADKDTCFSTEGPNLVTRCKDALA,
or
(SEQ ID NO: 20)
VEEPKNLVKTNCDLYEKLGEYGFQNAILVRYTQKAPQVSTPTLVEAARNL
GRVGTKCCTLPEDQRLPCVEDYLSAILNRVCLLHEKTPVSEHVTKCCSGS
LVERRPCFSALTVDETYVPKEFKAETFTFHSDICTLPEKEKQIKKQTALA
ELVKHKPKATAEQLKTVMDDFAQFLDTCCKAADKDTCFSTEGPNLVTRCK
DALA.

27. The polypeptide of claim 9, comprising the sequence of
(SEQ ID NO: 21)
MRFPSIFTAVLFAASSALAAPVNTTTEDETAQIPAEAVIGYSDLEGDFDV
AVLPFSNSTNNGLLFINTTIASIAAKEEGVSLEKRAGADDVVDSSKSFVM
ENFASYHGTKPGYVDSIQKGIQKPKSGTQGNYDDDWKGFYSTDNKYDAAG
YSVDNENPLSGKAGGVVKVTYPGLTKVLALKVDNAETIKKELGLSLTEPL
MEQVGTEEFIKRFGDGASRVVLSLPFAEGSSSVEYINNWEQAKALSVELE
INFETRGKRGQDAMYEYMAQACAGNRVRRSVGSSLSCINLDWDVIRDKTK
TKIESLKEHGPIKNKMSESPAKTVSEEKAKQYLEEFHQTALEHPELSELK
TVTGTNPVFAGANYAAWAVNVAQVIDSETADNLEKTTAALSILPGIGSVM
GIADGAVHHNTEEIVAQSIALSSLMVAQAIPLVGELVDIGFAAYNFVESI
INLFQVVHNSYNRPAYSPGHKTQPFLPWGGGASGAGTGSDIVMTQGTLPN
PVPSGESVSITCRSSKSLLYSDGKTYLNWYLQRPGQSPQLLIYWMSTRAS
GVSDRFSGSGSGTDFTLKISGVEAEDVGIYYCQQGLEFPTFGGGTKLELK
GGGGSGGGGSGGGGSEVQLQESGPGLVKPSQSLSLTCSVTGYSITSSYRW
NWIRKFPGNRLEWMGYINSAGISNYNPSLKRRISITRDTSKNQFFLQVNS
VTTEDAATYYCARSDNMGTTPFTYWGQGTLVTVSSGGGGSGGGGSGGGGS
DIVMTQGTLPNPVPSGESVSITCRSSKSLLYSDGKTYLNWYLQRPGQSPQ
LLIYWMSTRASGVSDRFSGSGSGTDFTLKISGVEAEDVGIYYCQQGLEFP
TFGGGTKLELKGGGGSGGGGSGGGGSEVQLQESGPGLVKPSQSLSLTCSV
TGYSITSSYRWNWIRKFPGNRLEWMGYINSAGISNYNPSLKRRISITRDT
SKNQFFLQVNSVTTEDAATYYCARSDNMGTTPFTYWGQGTLVTVSSGGGG
SGGGGSGGGGSHHHHHHHHHH.

28. The polypeptide of claim 9, comprising the sequence of
(SEQ ID NO: 22)
MRFPSIFTAVLFAASSALAAPVNTTTEDETAQIPAEAVIGYSDLEGDFDV
AVLPFSNSTNNGLLFINTTIASIAAKEEGVSLEKRAGADDVVDSSKSFVM
ENFASYHGTKPGYVDSIQKGIQKPKSGTQGNYDDDWKGFYSTDNKYDAAG
YSVDNENPLSGKAGGVVKVTYPGLTKVLALKVDNAETIKKELGLSLTEPL
MEQVGTEEFIKRFGDGASRVVLSLPFAEGSSSVEYINNWEQAKALSVELE
INFETRGKRGQDAMYEYMAQACAGNRVRRSVGSSLSCINLDWDVIRDKTK
TKIESLKEHGPIKNKMSESPAKTVSEEKAKQYLEEFHQTALEHPELSELK
TVTGTNPVFAGANYAAWAVNVAQVIDSETADNLEKTTAALSILPGIGSVM
GIADGAVHHNTEEIVAQSIALSSLMVAQAIPLVGELVDIGFAAYNFVESI
INLFQVVHNSYNRPAYSPGHKTQPFLPWGGGASGAGTGSDIVMTQGTLPN
PVPSGESVSITCRSSKSLLYSDGKTYLNWYLQRPGQSPQLLIYWMSTRAS
GVSDRFSGSGSGTDFTLKISGVEAEDVGIYYCQQGLEFPTFGGGTKLELK
GGGGSGGGGSGGGGSEVQLQESGPGLVKPSQSLSLTCSVTGYSITSSYRW
NWIRKFPGNRLEWMGYINSAGISNYNPSLKRRISITRDTSKNQFFLQVNS
VTTEDAATYYCARSDNMGTTPFTYWGQGTLVTVSSGGGGSGGGGSGGGGS
DIVMTQGTLPNPVPSGESVSITCRSSKSLLYSDGKTYLNWYLQRPGQSPQ
LLIYWMSTRASGVSDRFSGSGSGTDFTLKISGVEAEDVGIYYCQQGLEFP
TFGGGTKLELKGGGGSGGGGSGGGGSEVQLQESGPGLVKPSQSLSLTCSV
TGYSITSSYRWNWIRKFPGNRLEWMGYINSAGISNYNPSLKRRISITRDT
SKNQFFLQVNSVTTEDAATYYCARSDNMGTTPFTYWGQGTLVTVSSGGGG
SHHHHHH.

29. The polypeptide of claim 9, comprising the sequence of
(SEQ ID NO: 23)
MRFPSIFTAVLFAASSALAAPVNTTTEDETAQIPAEAVIGYSDLEGDFDV
AVLPFSNSTNNGLLFINTTIASIAAKEEGVSLEKRAGADDVVDSSKSFVM
ENFASYHGTKPGYVDSIQKGIQKPKSGTQGNYDDDWKGFYSTDNKYDAAG
YSVDNENPLSGKAGGVVKVTYPGLTKVLALKVDNAETIKKELGLSLTEPL
MEQVGTEEFIKRFGDGASRVVLSLPFAEGSSSVEYINNWEQAKALSVELE
INFETRGKRGQDAMYEYMAQACAGNRVRRSVGSSLSCINLDWDVIRDKTK
TKIESLKEHGPIKNKMSESPAKTVSEEKAKQYLEEFHQTALEHPELSELK
TVTGTNPVFAGANYAAWAVNVAQVIDSETADNLEKTTAALSILPGIGSVM
GIADGAVHHNTEEIVAQSIALSSLMVAQAIPLVGELVDIGFAAYNFVESI
INLFQVVHNSYNRPAYSPGHKTQPFLPWGGGASGAGTGSLAEAKVLANRE
LDKYGVSDFYKRLINKAKTVEGVEALKLHILAALPGGGGSGGGGSGTGSD
IVMTQGTLPNPVPSGESVSITCRSSKSLLYSDGKTYLNWYLQRPGQSPQL
LIYWMSTRASGVSDRFSGSGSGTDFTLKISGVEAEDVGIYYCQQGLEFPT
FGGGTKLELKGGGGSGGGGSGGGGSEVQLQESGPGLVKPSQSLSLTCSVT
GYSITSSYRWNWIRKFPGNRLEWMGYINSAGISNYNPSLKRRISITRDTS
KNQFFLQVNSVTTEDAATYYCARSDNMGTTPFTYWGQGTLVTVSSGGGGS
GGGGSGGGGSDIVMTQGTLPNPVPSGESVSITCRSSKSLLYSDGKTYLNW
YLQRPGQSPQLLIYWMSTRASGVSDRFSGSGSGTDFTLKISGVEAEDVGI
YYCQQGLEFPTFGGGTKLELKGGGGSGGGGSGGGGSEVQLQESGPGLVKP
SQSLSLTCSVTGYSITSSYRWNWIRKFPGNRLEWMGYINSAGISNYNPSL
KRRISITRDTSKNQFFLQVNSVTTEDAATYYCARSDNMGTTPFTYWGQGT
LVTVSSGGGGSGGGGSGGGGSHHHHHHHHHH.

30. The polypeptide of claim 9, comprising the sequence of
(SEQ ID NO: 24)
MRFPSIFTAVLFAASSALAAPVNTTTEDETAQIPAEAVIGYSDLEGDFDV
AVLPFSNSTNNGLLFINTTIASIAAKEEGVSLEKRAGADDVVDSSKSFVM
ENFASYHGTKPGYVDSIQKGIQKPKSGTQGNYDDDWKGFYSTDNKYDAAG
YSVDNENPLSGKAGGVVKVTYPGLTKVLALKVDNAETIKKELGLSLTEPL
MEQVGTEEFIKRFGDGASRVVLSLPFAEGSSSVEYINNWEQAKALSVELE
INFETRGKRGQDAMYEYMAQACAGNRVRRSVGSSLSCINLDWDVIRDKTK
TKIESLKEHGPIKNKMSESPAKTVSEEKAKQYLEEFHQTALEHPELSELK
TVTGTNPVFAGANYAAWAVNVAQVIDSETADNLEKTTAALSILPGIGSVM
GIADGAVHHNTEEIVAQSIALSSLMVAQAIPLVGELVDIGFAAYNFVESI
INLFQVVHNSYNRPAYSPGHKTQPFLPWGGGASGAGTGSLAEAKVLANRE
LDKYGVSDFYKRLINKAKTVEGVEALKLHILAALPGGGGSGGGGSGTGSD
IVMTQGTLPNPVPSGESVSITCRSSKSLLYSDGKTYLNWYLQRPGQSPQL
LIYWMSTRASGVSDRFSGSGSGTDFTLKISGVEAEDVGIYYCQQGLEFPT
FGGGTKLELKGGGGSGGGGSGGGGSEVQLQESGPGLVKPSQSLSLTCSVT
GYSITSSYRWNWIRKFPGNRLEWMGYINSAGISNYNPSLKRRISITRDTS
KNQFFLQVNSVTTEDAATYYCARSDNMGTTPFTYWGQGTLVTVSSGGGGS
GGGGSGGGGSDIVMTQGTLPNPVPSGESVSITCRSSKSLLYSDGKTYLNW
YLQRPGQSPQLLIYWMSTRASGVSDRFSGSGSGTDFTLKISGVEAEDVGI
YYCQQGLEFPTFGGGTKLELKGGGGSGGGGSGGGGSEVQLQESGPGLVKP
SQSLSLTCSVTGYSITSSYRWNWIRKFPGNRLEWMGYINSAGISNYNPSL
KRRISITRDTSKNQFFLQVNSVTTEDAATYYCARSDNMGTTPFTYWGQGT
LVTVSSGGGGSHHHHHH.

31. A nucleic acid construct comprising a nucleic acid sequence capable of encoding the polypeptide of claim 9.

32.-53. (canceled)

54. A composition comprising the polypeptide of claim 9.

55. The composition of claim 54, further comprising a pharmaceutically acceptable carrier

56. A method of treating a subject having an autoimmune disease, cancer, Type 1 Diabetes, or experimental autoimmune enchephalomyelitis comprising administering to the subject a composition comprising the polypeptide of claim 9.

57.-63. (canceled)

64. A method of killing PD-1 positive cells comprising contacting a PD-1 positive cell with a composition comprising the polypeptide of claim 9.

65. (canceled)
